(19) United States
(12) Patent Application Publication
(10) Pub. No.: US 2026/0183421 A1
(43) Pub. Date: Jul. 2, 2026

(54) Method for the manufacture of gastroretentive dosage forms

(71) Applicant: Aron H. Blaesi, Cambridge, MA (US)

(72) Inventors: Aron H. Blaesi, Cambridge, MA (US); Nannaji Saka, Cambridge, MA (US)

(73) Assignee: Aron H. Blaesi, Cambridge, MA (US)

(21) Appl. No.: 19/546,547

(22) Filed: Feb. 23, 2026

Related U.S. Application Data
(63) Continuation of application No. PCT/US2024/043312, filed on Aug. 21, 2024.
(63) Continuation-in-part of application No. 18/908,569, filed on Oct. 7, 2024.
Application No. 18/908,569 is a continuation-in-part of application No. PCT/US2024/043308, filed on Aug. 21, 2024.
(63) Continuation-in-part of application No. 19/546,338, filed on Feb. 21, 2026.
Application No. 19/546,338 is a continuation of application No. PCT/US2024/043307, filed on Aug. 21, 2024.
(63) Continuation-in-part of application No. 19/546,318, filed on Feb. 21, 2026.
Application No. 19/546,318 is a continuation of application No. PCT/US2024/043309, filed on Aug. 21, 2024.
(60) Provisional application No. 63/533,792, filed on Aug. 21, 2023.

(51) Int. Cl. A61K 47/69; A61K 31/506; A61K 47/02; A61K 47/32; A61K 47/38

(52) U.S. Cl. CPC A61K 47/6953; A61K 31/506; A61K 47/02; A61K 47/32; A61K 47/38

(57) ABSTRACT

In recently or concurrently filed disclosures, we have presented expandable, gastroretentive dosage forms with independently controllable gastric residence time and drug release rate. In this specification, a method for producing such and similar dosage forms is disclosed. The method comprises preparing a three-dimensional structural framework of one or more fibers, and attaching a controlled amount of drug-containing matter to said three-dimensional fiber structural framework.

### CROSS-REFERENCE TO RELATED INVENTIONS

This application is a continuation of, and incorporates herein by reference in its entirety, the International Application No. PCT/US2024/043312 filed on Aug. 21, 2024 and titled “Method for the manufacture of gastroretentive dosage form”, which claims priority to and the benefit of the U.S. Provisional Application No. U.S. 63/533,792 filed on Aug. 21, 2023. Also the foregoing Provisional Application is incorporated herein by reference in its entirety.

This application is a continuation-in-part of, and incorporates herein by reference in its entirety, the U.S. application Ser. No. 18/908,569 filed on Oct. 7, 2024 and titled “Gastroretentive fibrous dosage form for prolonged drug delivery”, which is a continuation-in-part of the International Application No. PCT/US2024/043308 filed on Aug. 21, 2024 and titled “Gastroretentive fibrous dosage form for prolonged drug delivery”. Also the foregoing International Application is incorporated herein by reference in its entirety.

This application is also a continuation-in-part of, and incorporates herein by reference in its entirety, the U.S. application Ser. No. 19/546,318 filed on Feb. 21, 2026 and titled “Fluid-absorptive gastroretentive dosage form for prolonged drug delivery”, which is a continuation of the International Application No. PCT/US2024/043309 filed on Aug. 21, 2024 and titled “Fluid-absorptive gastroretentive dosage form for prolonged drug delivery”. Also the foregoing International Application is incorporated herein by reference in its entirety.

This application is also a continuation-in-part of, and incorporates herein by reference in its entirety, the U.S. application Ser. No. 19/546,338 filed on Feb. 21, 2026 and titled “Gastroretentive dosage form for prolonged drug delivery”, which is a continuation of the International Application No. PCT/US2024/043307 filed on Aug. 21, 2024 and titled “Gastroretentive dosage form for prolonged drug delivery”. Also the foregoing International Application is incorporated herein by reference in its entirety.

This application is related to and incorporates herein by reference in their entirety, the U.S. application Ser. No. 15/482,776 filed on Apr. 9, 2017 and titled “Fibrous dosage form”, the U.S. application Ser. No. 15/964,058 filed on Apr. 26, 2018 and titled “Method and apparatus for the manufacture of fibrous dosage forms”, the U.S. application Ser. No. 16/860,911 filed on Apr. 28, 2020 and titled “Expandable structured dosage form for immediate drug delivery”, the U.S. application Ser. No. 16/916,208 filed on Jun. 30, 2020 and titled “Dosage form comprising structural framework of two-dimensional elements”, the U.S. application Ser. No. 17/237,034 filed on Apr. 21, 2021 and titled “Method for 3D-micro-patterning”, the U.S. application Ser. No. 17/327,721 filed on May 23, 2021 and titled “Expandable multi-excipient dosage form”, and the U.S. application Ser. No. 18/124,381 filed on Mar. 21, 2023 and titled “Gastroretentive structured dosage form”, the International Application No. PCT/US19/19004 filed on Feb. 21, 2019 and titled “Expanding structured dosage form”, the International Application No. PCT/US19/52030 filed on Sep. 19, 2019 and titled “Dosage form comprising structured solid-solution framework of sparingly-soluble drug and method for manufacture thereof”, the International Application No. PCT/US21/22857 filed on Mar. 17, 2021 and titled “Expandable, multi-excipient structured dosage form for prolonged drug release”, and the International Application No. PCT/US21/22860 filed on Mar. 17, 2021 and titled “Method and apparatus for 3D-micro-patterning”.

### BACKGROUND OF THE INVENTION

In recently or concurrently filed disclosures, the present inventors (Blaesi and Saka) have introduced pharmaceutical solid dosage forms comprising a drug-containing solid attached to a three-dimensional fibrous structural network (see, e.g., the U.S. application Ser. No. 18/908,569 titled “Gastroretentive fibrous dosage form for prolonged drug delivery”, the U.S. application Ser. No. 19/546,318 titled “Fluid-absorptive gastroretentive dosage form for prolonged drug delivery”, or the U.S. application Ser. No. 19/546,338 titled “Gastroretentive dosage form for prolonged drug delivery”). In this disclosure, a method for producing such and similar dosage forms is presented.

### SUMMARY OF THE INVENTION

Generally, in the invention herein a drug-laden formulation may be applied outside an expandable solid (e.g., water-absorbing fibers coated with a strengthening, enteric excipient, etc.).

More specifically, in one aspect a method of manufacturing pharmaceutical solid dosage forms disclosed herein comprises the steps of: preparing a supporting solid comprising an outer surface and an internal, three dimensional support structure contiguous with and terminating at said outer surface; and adding or attaching a controlled amount of drug-containing matter to said support structure.

In some embodiments, the support structure comprises a supporting three-dimensional structural framework of one or more elements.

In some embodiments, moreover, the support structure comprises a supporting three-dimensional structural framework of one or more fibers.

In another aspect, therefore, a method of manufacturing pharmaceutical solid dosage forms herein comprises the steps of: preparing a supporting three-dimensional structural framework of one or more fibers; and adding or attaching a controlled amount of drug-containing matter to said three-dimensional fiber structural framework.

In some embodiments, adding or attaching a controlled amount of drug-containing matter to a supporting three-dimensional fiber structural framework comprises the steps of: forming a drug-containing plasticized matrix, said drug-containing plasticized matrix disposed in an extrusion channel and comprising at least one drug and at least one second pharmaceutical excipient; extruding said drug-containing plasticized matrix through said extrusion channel by application of mechanical work; filling a controlled amount of said extruded drug-containing plasticized matrix into one or more free spaces between fibers or fiber segments of said supporting fiber structural framework; and solidifying said drug-containing plasticized matrix within said one or more free spaces to form drug-containing solid attached to said three-dimensional fiber structural framework.

In another aspect, therefore, a method of manufacturing pharmaceutical solid dosage forms herein comprises the steps of: preparing a supporting three-dimensional structural framework of one or more fibers, said one or more fibers comprising at least a first pharmaceutical excipient; forming a drug-containing plasticized matrix, said drug-containing plasticized matrix disposed in an extrusion channel and comprising at least one drug and at least one second pharmaceutical excipient; extruding said drug-containing plasticized matrix through said extrusion channel by application of mechanical work; filling a controlled amount of said extruded drug-containing plasticized matrix into one or more free spaces between fibers or fiber segments of said supporting fiber structural framework; and solidifying said drug-containing plasticized matrix within said one or more free spaces to form drug-containing solid attached to said three-dimensional fiber structural framework.

In some embodiments, preparing the supporting three-dimensional structural framework of one or more fibers comprises the steps of: forming a plasticized matrix comprising at least a first pharmaceutical excipient, said plasticized matrix disposed in an extrusion channel; extruding said plasticized matrix through a fiber fabrication exit port of said extrusion channel by application of mechanical work to form extruded plasticized fiber; depositing said extruded plasticized fiber onto a fiber assembling stage to form a three-dimensional fiber structural framework defined by the motion of said stage; and solidifying said three-dimensional fiber structural framework.

In some embodiments, the plasticized matrix comprising at least a first pharmaceutical excipient is formed by heating a composition comprising at least one first pharmaceutical excipient.

In some embodiments, the plasticized matrix comprising at least a first pharmaceutical excipient is formed by mixing at least a first pharmaceutical excipient and at least a first solvent solvating said first excipient.

In some embodiments, mechanical work to extrude plasticized matrix through an exit port or a fiber fabrication exit port is applied on said plasticized matrix by one or more rotating screws.

In some embodiments, mechanical work to extrude plasticized matrix through an exit port or fiber fabrication exit port is applied on said plasticized matrix by an advancing piston.

In some embodiments, plasticized matrix is extruded through an exit port or a fiber fabrication exit port at a controlled speed.

In some embodiments, plasticized matrix is extruded through an exit port or a fiber fabrication exit port at a speed determined by the speed of advancement of a piston.

In some embodiments, extruded plasticized fiber or deposited three-dimensional fiber structural framework is solidified by cooling or by evaporating at least a first solvent.

In some embodiments, the drug-containing plasticized matrix is formed by heating a composition comprising at least one second pharmaceutical excipient.

In some embodiments, the drug-containing plasticized matrix is formed by mixing at least a second pharmaceutical excipient and at least a solvent (e.g., a second solvent) solvating said second excipient.

In some embodiments, mechanical work to extrude drug-containing plasticized matrix through said extrusion channel is applied on said plasticized matrix by one or more rotating screws.

In some embodiments, mechanical work to extrude drug-containing plasticized matrix through said extrusion channel is applied on said plasticized matrix by an advancing piston.

In some embodiments, drug-containing plasticized matrix is extruded through said extrusion channel at a controlled speed.

In some embodiments, drug-containing plasticized matrix is extruded through said extrusion channel at a speed determined by the speed of advancement of a piston.

In some embodiments, drug-containing plasticized matrix is solidified by cooling or by evaporating at least a solvent (e.g., a second solvent).

In some embodiments, a method herein further comprises the step of coating a supporting three-dimensional structural framework of one or more fibers or a part thereof with a coating,

In some embodiments, a coating comprises at least a third pharmaceutical excipient.

In some embodiments, a method herein further comprises the step of pushing a needle or pin into or through drug-containing matter to form one or more channels within said drug-containing matter.

In some embodiments, the three-dimensional structural framework of one or more fibers is prepared by 3D-micro-patterning or 3D-printing.

In another aspect, a method of manufacturing pharmaceutical solid dosage forms herein comprises the steps of: preparing a supporting three-dimensional structural framework of one or more fibers, said one or more fibers comprising at least a first pharmaceutical excipient; and adding or attaching a controlled amount of drug-containing matter to said supporting three-dimensional structural framework; wherein the step of preparing the supporting comprises the steps of: forming a plasticized matrix comprising at least a first pharmaceutical excipient, said plasticized matrix disposed in an extrusion channel; extruding said plasticized matrix through a fiber fabrication exit port of said extrusion channel by application of mechanical work to form extruded plasticized fiber; depositing said extruded plasticized fiber onto a fiber assembling stage to form a three-dimensional fiber structural framework defined by the motion of said stage; and solidifying said three-dimensional fiber structural framework; and wherein the step of adding or attaching a controlled amount of drug-containing matter to said supporting three-dimensional structural framework of one or more fibers comprises the steps of: forming a drug-containing plasticized matrix, said drug-containing plasticized matrix disposed in an extrusion channel and comprising at least one drug and at least one second pharmaceutical excipient; extruding said drug-containing plasticized matrix through said extrusion channel by application of mechanical work; filling a controlled amount of said extruded drug-containing plasticized matrix into one or more free spaces between fibers or fiber segments of said supporting fiber structural framework; and solidifying said drug-containing plasticized matrix within said one or more free spaces to form drug-containing solid attached to said three-dimensional fiber structural framework.

### BRIEF DESCRIPTION OF THE DRAWINGS

The objects, embodiments, features, and advantages of the present invention are more fully understood when considered in conjunction with the following accompanying drawings:

FIG. 1 presents a non-limiting schematic of a method of manufacturing pharmaceutical solid dosage forms according to this invention. The method comprises the steps of (a) preparing a supporting solid comprising an outer surface and an internal, three dimensional support structure contiguous with and terminating at said outer surface, and (b) adding drug-containing matter to said support structure;

FIGS. 2-11 present additional non-limiting methods of manufacturing pharmaceutical solid dosage forms according to the invention herein;

FIGS. 12-16 present non-limiting methods of manufacturing supporting solids herein;

FIGS. 17-20 present non-limiting methods of coating support structures of supporting solids herein;

FIGS. 21-26 present non-limiting methods of adding drug-containing matter to support structures herein; and

FIG. 27 depicts scanning electron micrographs of fibrous dosage forms according to this invention: (a) top view and (b) longitudinal section of uncoated fibrous cylindrical disk; (c) top view and (d) longitudinal section of coated fibrous cylindrical disk; (e) top view and (f) cross section of “final” dosage form.

### DEFINITIONS

In order for the present disclosure to be more readily understood, certain terms are first defined below. Additional definitions for the following terms and other terms are set forth throughout the specification.

In this application, the use of “or” means “and/or” unless stated otherwise. As used in this application, the term “comprise” and variations of the term, such as “comprising” and “comprises” are not intended to exclude other additives, components, integers or steps. As used in this application, the terms “about” and “approximately” are used as equivalents. Any numerals used in this application with or without about/approximately are meant to cover any normal fluctuations appreciated by one of ordinary skill in the relevant art.

Moreover, in the disclosure herein, the terms “one or more active ingredients”, “active ingredient”, “active pharmaceutical ingredient”, and “drug” are used interchangeably. As used herein, an “active ingredient” or “active agent” refers to an agent whose presence or level correlates with elevated level or activity of a target, as compared with that observed absent the agent (or with the agent at a different level). In some embodiments, an active ingredient is one whose presence or level correlates with a target level or activity that is comparable to or greater than a particular reference level or activity (e.g., that observed under appropriate reference conditions, such as presence of a known active agent, e.g., a positive control).

In the invention herein, a “solid” or “solid material” is typically referred to an elastic or viscoelastic material. Such elastic or viscoelastic materials may be characterized by a very large viscosity that may be far greater than about 10⁵ Pa·s. This includes, but is not limited to a viscosity greater than 10⁷ Pa·s, or greater than 10¹⁰ Pa·s. In the limiting case, the viscosity is so large that it is difficult to measure. A solid material may be considered “fully elastic” or “elastic” in this case. For further details about the mechanical behavior of elastic or visco-elastic materials, see, e.g., K. L. Johnson, “Contact mechanics”, Cambridge University Press, 1985.

In the context of the invention herein, a plasticized matrix generally is a viscous material comprising a shear viscosity in the range of about 0.01 Pa·s-5×10⁸ Pa·s at a shear rate no greater than about 10 l/s. This includes but is not limited to a minimum shear viscosity of 0.01 Pa·s-10⁸, 0.01 Pa·s-5×10⁷ Pa·s, 0.025 Pa·s-10,000,000 Pa·s, 0.05 Pa·s-5,000,000 Pa·s, 0.1 Pa·s-2,000,000 Pa·s, 0.25 Pa·s-1,000,000 Pa·s, 0.25 Pa·s-5,000,000 Pa·s, 0.5 Pa·s-2,000,000 Pa·s, 1 Pa·s-2,000,000 Pa·s, 1 Pa·s-5,000,000 Pa·s, 1 Pa·s-1,000,000 Pa·s, 10 Pa·s-1,000,000 Pa·s, 50 Pa·s-1,000,000 Pa·s, 100 Pa·s-1,000,000 Pa·s, or 1 Pa·s-500,000 Pa·s at a shear rate no greater than about 10 l/s. Non-limiting examples of plasticized matrices include but are not limited to polymer melts, concentrated solutions of one or more polymers and one or more solvents (e.g., water, ethanol, acetone, isopropanol, ethyl acetate, dimethyl sulfoxide, etc.), suspensions of solid particulates or granules and a polymer melt, suspensions of solid particulates and a concentrated polymeric solution, etc. It may be noted that in the context of the invention herein the terms “plasticized matrix”, “plasticized matrices”, “plasticized material”, and “melt” are used interchangeably. Furthermore, in some embodiments a plasticized matrix may include an active ingredient. The active ingredient may be molecularly dissolved in the plasticized matrix, dispersed as particles in the plasticized matrix, and so on.

In the invention herein, an internal, three dimensional support structure is generally referred to a solid or solid material, including non-porous solids, minimally-porous solids, porous solids, cellular solids, solid framework structures, solid network structures, solid lattice structures, and so on. The term “internal, three dimensional support structure” is used interchangeably with “internal support structure”, “three-dimensional support structure”, “support structure”, “three dimensional structural framework of one or more elements”, and so on.

Furthermore, in the context of some embodiments herein, a three dimensional structural framework of one or more elements may comprise a structure (e.g., a framework of one or more elements, an assembly or an assemblage of one or more elements, an arrangement of one or more elements, a skeleton or skeletal structure of one or more elements, a three-dimensional network or network structure of one or more elements, a three-dimensional lattice structure of one or more elements, etc.) that extends over a length, width, and thickness greater than 100 μm. This includes, but is not limited to structures that extend over a length, width, and thickness greater than 200 μm, or greater than 300 μm, or greater than 500 μm, or greater than 700 μm, or greater than 1 mm, or greater than 1.25 mm, or greater than 1.5 mm, or greater than 2 mm.

In some embodiments, moreover, a three dimensional structural framework of one or more elements may comprise a structure (e.g., a framework of one or more elements, an assembly or an assemblage of one or more elements, an arrangement of one or more elements, a skeleton or skeletal structure of one or more elements, a three-dimensional network or network structure of one or more elements, a three-dimensional lattice structure of one or more elements, etc.) that extends over a length, width, and thickness greater than the average thickness of at least one element (or at least one segment) in the three dimensional structural framework (or network) of elements. This includes, but is not limited to structures that extend over a length, width, and thickness greater than 1.5, or greater than 2, or greater than 2.5, or greater than 3, or greater than 3.5, or greater than 4 times the average thickness of at least one element (or at least one segment) in the three dimensional structural framework (or network) of elements.

In the invention herein, the terms “three dimensional structural framework of one or more elements”, “three dimensional structural framework of elements”, “structural framework of one or more elements”, “structural framework of elements”, “framework of one or more elements”, “framework of elements”, “structural framework”, “framework”, “three dimensional structural framework of fibers”, “three dimensional structural framework of one or more fibers”, “three dimensional structural framework of fibers”, and so on are used interchangeably.

Moreover, it may be noted that the terms “three dimensional structural network”, “structural network”, “three dimensional structural framework”, “structural framework”, “framework”, “three dimensional lattice structure”, and so on are used interchangeably herein.

In some embodiments, a three dimensional structural framework (or network) of elements may be continuous.

In the invention herein, a “structural element” or “element” may generally refer to a two-dimensional element (or 2-dimensional structural element), or a one-dimensional element (or 1-dimensional structural element), or a zero-dimensional element (or 0-dimensional structural element).

As used herein, a two-dimensional structural element may generally be referred to as having a length and width much greater than its thickness. In the present disclosure, the length and width of a two-dimensional structural element generally are greater than 2 times its thickness. An example of such an element is a “sheet”. A one-dimensional structural element generally is referred to as having a length much greater than its width and thickness. In the present disclosure, the length of a one-dimensional structural element generally is greater than 2 times its width and thickness. An example of such an element is a “fiber”. A zero-dimensional structural element generally is referred to as having a length and width of the order of its thickness. In the present disclosure, the length and width of a zero-dimensional structural element are no greater than 2 times its thickness. Furthermore, the thickness of a zero-dimensional element generally is less than 2.5 mm. Examples of such zero-dimensional elements are “particles” or “beads” and include polyhedra, spheroids, ellipsoids, or clusters thereof.

Moreover, in the invention herein, a segment of a one-dimensional element may generally be referred to as a fraction of said element along its length. A segment of a two-dimensional element may generally be referred to as a fraction of said element along its length and/or width. A segment of a zero-dimensional element may generally be referred to as a fraction of said element along its length and/or width and/or thickness. The terms “segment of a one-dimensional element”, “fiber segment”, “segment of a fiber”, and “segment” are used interchangeably herein. Also, the terms “segment of a two-dimensional element” and “segment” are used interchangeably herein. Also, the terms “segment of a zero-dimensional element” and “segment” are used interchangeably herein.

Furthermore, in some embodiments, one or more elements or segments thereof may be bonded to each other. In some embodiments, moreover, one or more elements or segments thereof may be interpenetrating.

As used herein, moreover, the terms “fiber”, “fibers”, and “one or more fibers”, are used interchangeably. Fibers are generally understood as one-dimensional elements. Thus, a fiber may have a length much greater than its width and thickness. In the present disclosure, a fiber may generally be referred to as having a length greater than 2 times its width and thickness (e.g., the length may be greater than 2 times the fiber width and the length may be greater than 2 times the fiber thickness). This includes, but is not limited to a fiber length greater than 3 times, or greater than 4 times, or greater than 5 times, or greater than 6 times, or greater than 8 times, or greater than 10 times, or greater than 12 times the fiber width and thickness. In other embodiments that are included but not limiting in the disclosure herein, the length of a fiber may be greater than 0.3 mm, or greater than 0.5 mm, or greater than 1 mm, or greater than 2.5 mm.

Moreover, as used herein, fibers can be either solid or plasticized. The terms “plasticized fiber” and “wet fiber” are used interchangeably herein. Plasticized and wet fibers are understood as viscous fibers with a viscosity of the order of the viscosity of the plasticized matrix from which they were formed.

Moreover, as used herein, the term “fiber segment” or “segment” may generally refer to a fraction of a fiber along the length of said fiber.

In the invention herein, fibers (or fiber segments) may be bonded, and thus they may serve as building blocks of “assembled structural elements” with a geometry different from that of the original fibers. Such assembled structural elements include two-dimensional elements, one-dimensional elements, or zero-dimensional elements.

In the invention herein, any three dimensional structural framework comprising at least one structural element (e.g., a zero-dimensional, one-dimensional, or two-dimensional structural element) comprising an arrangement/assembly/assemblage of bonded fibers or bonded fiber segments is considered a three dimensional structural network of one or more fibers.

In the invention herein, moreover, the term drug-containing matter is generally referred to a drug-containing material or a material that includes at least a drug. By way of example but not by way of limitation, drug-containing matter can be a drug-containing solid, a drug-containing plasticized matrix, a combination of drug-containing solid and drug-containing plasticized matrix, a drug-containing solution, a drug-containing dispersion, and so on.

In the invention herein, drug release from a drug-containing solid (or a drug releasable solid, or a solid dosage form, or a pharmaceutical solid dosage form, etc.) refers to the conversion of drug (e.g., one or more drug particles, or drug molecules, or clusters thereof, etc.) that is/are embedded in or attached to the drug-containing solid (or a drug releasable solid, or a solid dosage form, or a pharmaceutical solid dosage form, etc.) to drug in a dissolution medium.

Further information related to the definition, characteristics, features, composition, analysis etc. of the disclosed dosage forms, and the elements for fabricating or constructing them, is provided throughout this specification.

### Scope of the Invention

It is contemplated that a particular feature described either individually or as part of an embodiment in this disclosure can be combined with other individually described features, or parts of other embodiments, even if the other features and embodiments make no mention of the particular feature. Thus, the invention herein extends to such specific combinations not already described. Furthermore, the drawings and embodiments of the invention herein have been presented as examples, and not as limitations. Thus, it is to be understood that the invention herein is not limited to these precise embodiments. Other embodiments apparent to those of ordinary skill in the art are within the scope of what is claimed.

By way of example but not by way of limitation, it is contemplated that compositions, systems, devices, methods, and processes of the claimed invention encompass variations and adaptations developed using information from the embodiments described herein. Adaptation and/or modification of the compositions, systems, devices, methods, and processes described herein may be performed by those of ordinary skill in the relevant art.

Furthermore, where compositions, articles, and devices are described as having, including, or comprising specific components, or where processes and methods are described as having, including, or comprising specific steps, it is contemplated that, additionally, there are compositions, articles, and devices of the present invention that consist essentially of, or consist of, the recited components, and that there are processes and methods according to the present invention that consist essentially of, or consist of, the recited processing steps.

Similarly, where compositions, articles, and devices are described as having, including, or comprising specific compounds and/or materials, it is contemplated that, additionally, there are compositions, articles, and devices of the present invention that consist essentially of, or consist of, the recited compounds and/or materials.

It should be understood that the order of steps or order for performing certain action is immaterial so long as the invention remains operable. Moreover, two or more steps or actions may be conducted simultaneously.

The mention herein of any publication is not an admission that the publication serves as prior art with respect to any of the claims presented herein. Headers are provided for organizational purposes and are not meant to be limiting.

### DETAILED DESCRIPTION OF THE INVENTION

### Aspects and Embodiments of the Disclosed Method of Manufacturing Pharmaceutical Solid Dosage Forms

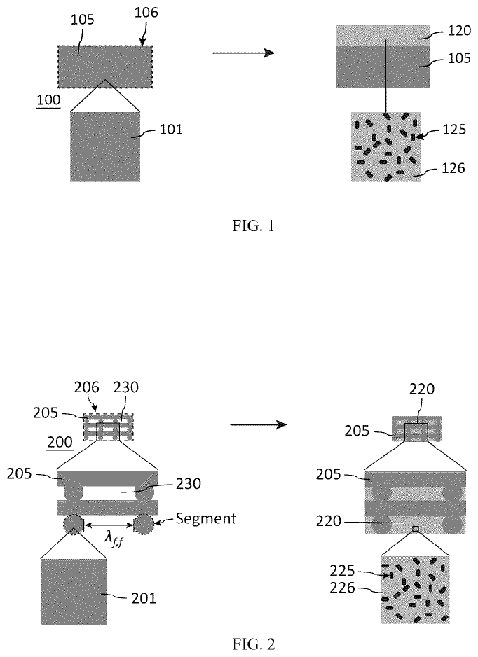

FIG. 1 presents anon-limiting method of manufacturing pharmaceutical solid dosage forms according to the invention herein. The method comprises the steps of (a) preparing (e.g., manufacturing) a supporting solid 100 comprising an outer surface 106 and an internal, three dimensional support structure 105 contiguous with and terminating at said outer surface 106, and (b) adding or attaching (e.g., immovably attaching to, bonding to, adhering to, partially or entirely surrounding, partially or entirely covering, attaching to a surface of, partially or entirely covering a surface of, etc.) drug-containing matter 120 to said support structure 105. It may be noted that the term “adding or attaching drug-containing matter 120 to said support structure 105” includes, but is not limited to “adding or attaching drug-containing matter 120 to a surface or a deposition surface of said support structure 105”, and so on.

In the invention herein, any support structure (e.g., an internal support structure, an internal, three-dimensional support structure, a three dimensional structural framework of one or more elements, a three dimensional structural framework of one or more fibers, etc.), may generally comprise at least a first pharmaceutical excipient 101. Said first pharmaceutical excipient 101 may generally comprise any material suitable for oral ingestion by a human or animal subject. Preferably, moreover, said first pharmaceutical excipient 101 may be solid at room temperature. Said first pharmaceutical excipient 101 is also referred to herein as “first excipient”.

Generally, moreover, drug-containing matter 120 herein may include at least an active pharmaceutical ingredient or drug 125. Preferably, moreover, drug-containing matter 120 may further comprise at least a second pharmaceutical excipient 126. Said second pharmaceutical excipient 126, too, may generally comprise any material suitable for oral ingestion by a human or animal subject. Preferably, moreover, said second pharmaceutical excipient 126 may be solid at room temperature. Preferably, furthermore, said second pharmaceutical excipient may allow the release of drug upon immersing said drug-containing matter 120 in a physiological fluid under physiological conditions. Said second pharmaceutical excipient 126 is also referred to herein as “second excipient”.

In some embodiments, moreover, an internal, three dimensional support structure may comprise a three dimensional structural framework of one or more structural elements.

FIG. 2 presents a non-limiting method of manufacturing pharmaceutical solid dosage forms herein that includes such embodiments. The method comprises the steps of (a) preparing (e.g., manufacturing) a supporting solid 200 comprising an outer surface 206 and an internal, three dimensional structural framework of one or more elements 205, said structural framework of elements 205 contiguous with and terminating at said outer surface 206, and (b) adding or attaching (e.g., immovably attaching to, bonding to, adhering to, partially or entirely surrounding, partially or entirely covering, attaching to a surface of, partially or entirely covering a surface of, etc.) drug-containing matter 220 to said three-dimensional structural framework of one or more elements 205. It may be noted that the term “adding or attaching drug-containing matter 220 to said three-dimensional structural framework of one or more elements 205” includes, but is not limited to “adding or attaching drug-containing matter 220 to a surface or a deposition surface of said three-dimensional structural framework of one or more elements 205”, and so on.

In the invention herein, the terms “support structure”, “internal support structure”, “three-dimensional structural framework of one or more elements”, “three-dimensional structural framework of elements”, “three dimensional support structure”, and “internal, three dimensional support structure” are used interchangeably.

In some embodiments, moreover, an internal, three dimensional support structure may comprise a three dimensional structural framework of one or more fibers.

FIG. 2 presents a non-limiting method of manufacturing pharmaceutical solid dosage forms herein that includes such embodiments. The method comprises the steps of (a) preparing (e.g., manufacturing) a supporting solid 200 comprising an outer surface 206 and an internal, three dimensional structural framework of one or more fibers 205, said fibrous structural framework 205 contiguous with and terminating at said outer surface 206, and (b) adding or attaching (e.g., immovably attaching to, bonding to, adhering to, partially or entirely surrounding, partially or entirely covering, attaching to a surface of, partially or entirely covering a surface of, etc.) drug-containing matter 220 to said three-dimensional fiber structural framework 205. It may be noted that the term “adding or attaching drug-containing matter 220 to said three-dimensional fiber structural framework 205” includes, but is not limited to “adding or attaching drug-containing matter 220 to a surface or a deposition surface of said three-dimensional fiber structural framework 205”, and so on.

In the invention herein, moreover, the step of “preparing (e.g., manufacturing) a supporting solid 200 comprising an outer surface 206 and an internal, three dimensional structural framework of one or more fibers 205, said fibrous structural framework 205 contiguous with and terminating at said outer surface 206” may generally be understood as equivalent to “preparing (e.g., manufacturing) a supporting three-dimensional structural framework of one or more fibers 205”.

Accordingly, FIG. 2 also presents a non-limiting method of manufacturing pharmaceutical solid dosage forms comprising the steps of (a) preparing (e.g., manufacturing) a supporting three-dimensional structural framework of one or more fibers 205, and (b) adding or attaching a controlled amount of drug-containing matter 220 to said supporting fibrous structural framework. It may be noted that the term “adding or attaching a controlled amount of drug-containing matter 220 to said supporting fibrous structural framework 205” includes, but is not limited to “adding or attaching a controlled amount of drug-containing matter 220 to a surface or a deposition surface of said supporting fibrous structural framework 205”, and so on.

It may be noted, furthermore, that in any embodiment herein a three dimensional structural framework of one or more fibers 205 (or a three-dimensional structural framework of one or more elements, etc.) may generally comprise at least a first pharmaceutical excipient 201. Similarly, in any embodiment herein a drug-containing matter 220 may comprise at least a drug 225 and at least a second pharmaceutical excipient 226.

Generally, moreover, one or more fibers or one or more fiber segments in a supporting three dimensional fibrous structural framework 205 may be spaced apart from adjoining fibers or fiber segments by fiber-free spacings, lff, defining one or more fiber-free spaces 230 within an outer volume of said fibrous structural framework 205. In the invention herein, an “outer volume of a fibrous structural framework” may generally be understood as “envelope” of said “fibrous structural framework”.

Thus, in the invention herein the step of “preparing a supporting three-dimensional structural framework of one or more fibers 205” may generally be understood as equivalent to “preparing a supporting solid 200 comprising an outer surface 206, an internal, three dimensional structural framework of one or more fibers 205 contiguous with and terminating at said outer surface 206, and one or more free spaces 230 (e.g., one or more fiber-free spaces)”.

Generally, one or more fiber-free spaces 230 (e.g., one or more free spaces) may be substantially open to an exterior surface 206 of a fibrous structural framework 205 (e.g., an exterior surface 206 of a supporting solid 200). In other words, one or more fiber-free spaces 230 (e.g., one or more free spaces) may generally have at least one open end contiguous with and terminating at an exterior or outer surface 206 of a fibrous structural framework 205 (e.g., an exterior or outer surface of a supporting solid 200).

Thus, in the invention herein the step of “preparing a supporting three-dimensional structural framework of one or more fibers 205” may generally also be understood as equivalent to “preparing a supporting solid comprising an outer surface 206, an internal, three dimensional structural framework of one or more fibers 205 contiguous with and terminating at said outer surface 206, and one or more open free spaces 230 (e.g., one or more open fiber-free spaces)”.

In some embodiments, a fiber-free space 230 (or a free space) that is open may be filled with drug-containing matter 220 upon adding or attaching a controlled amount of drug-containing matter 220 to a supporting fiber structural framework 205 (or to a support structure). In some embodiments, moreover, drug-containing matter 220 may comprise a plasticized matrix. This includes, but is not limited to embodiments where drug-containing matter comprises a plasticized matrix that may be solidified to form a drug-containing solid.

Thus, by way of example but not by way of limitation, in some embodiments the step of “adding or attaching a controlled amount of drug-containing matter to a supporting fiber structural framework (or to a support structure)” may comprise “filling a controlled amount of a drug-containing plasticized matrix into one or more fiber-free spaces (or into one or more free spaces)”, and “solidifying said drug-containing plasticized matrix to form drug-containing solid added or attached to said three-dimensional fiber structural framework (or to said support structrure)”.

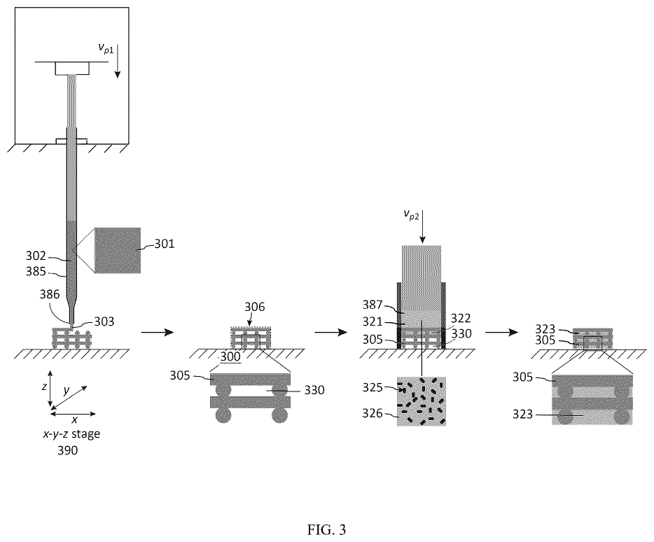

FIG. 3 presents anon-limiting example of a method of manufacturing pharmaceutical solid dosage forms herein that includes some of the above embodiments. The method comprises the steps of (a) preparing a supporting three-dimensional structural framework of one or more fibers 305, (b) filling (e.g., extruding, squeezing, transporting, etc.) a controlled amount of a drug-containing plasticized matrix 322 into one or more free spaces 330 between fibers or fiber segments of said supporting fiber structural framework 305, and (c) solidifying said drug-containing plasticized matrix 322 (e.g., the added drug-containing plasticized matrix 322) to form drug-containing solid 323 added or attached to said supporting three-dimensional fiber structural framework 305.

It may be noted that the steps of “filling (e.g., extruding, squeezing, transporting, etc.) a controlled amount of a drug-containing plasticized matrix 322 into one or more free spaces 330 between fibers or fiber segments of a supporting fiber structural framework 305” and “filling a controlled amount of extruded drug-containing plasticized matrix 322 into one or more free spaces 330 between fibers or fiber segments of a supporting fiber structural framework 305 to add drug-containing plasticized matrix 322 to said fiber structural framework 305” are used interchangeably herein. Also, the steps of “solidifying drug-containing plasticized matrix 322 to form drug-containing solid 323 added or attached to a supporting three-dimensional fiber structural framework 305” and “solidifying added drug-containing plasticized matrix 322 to form drug-containing solid 323 added or attached to a supporting three-dimensional fiber structural framework 305” are used interchangeably herein.

FIG. 3 also presents another non-limiting example of a method of manufacturing pharmaceutical solid dosage forms according to this invention. The method comprises the steps of (a) preparing a supporting solid 300 comprising an exterior or outer surface 306, an internal, three-dimensional solid support structure 305 contiguous with and terminating at said outer surface 306, and one or more internal, open free spaces 330, (b) filling (e.g., extruding, squeezing, transporting, etc.) a controlled amount of a drug-containing plasticized matrix 322 into one or more internal, open free spaces 330, and (c) solidifying said drug-containing plasticized matrix 322 to form drug-containing solid 323 added or attached to said support structure 305.

It may be noted that the steps of “filling (e.g., extruding, squeezing, transporting, etc.) a controlled amount of a drug-containing plasticized matrix 322 into one or more internal, open free spaces 330” and “filling a controlled amount of a drug-containing plasticized matrix 322 into one or more free spaces 330 of a supporting solid 300 to add drug-containing plasticized matrix 322 to a support structure 305 of said supporting solid 300” are used interchangeably herein. Also, the steps of “solidifying drug-containing plasticized matrix 322 to form drug-containing solid 323 added or attached to a support structure 305” and “solidifying added drug-containing plasticized matrix 322 to form drug-containing solid 323 attached to a support structure 305” are used interchangeably herein.

In some embodiments, moreover, a supporting three dimensional fiber structural framework 305 (or a three dimensional structural framework of one or more elements, or a support structure of a supporting solid, etc.) may be prepared (e.g., manufactured, produced, etc.) by 3D-printing, 3D-micro-patterning, 3D-patterning, patterning, printing, and so on.

FIG. 3 also presents a non-limiting example of a method of manufacturing pharmaceutical solid dosage forms herein that includes some of these embodiments. The method comprises the steps of (a) extruding a plasticized matrix 302 comprising at least a first pharmaceutical excipient 301 through an exit port 386 of an extrusion channel 385 by application of mechanical work to form plasticized extrudate 303 (e.g., fibrous plasticized extrudate) (b) depositing (e.g., patterning, printing, etc.) said plasticized extrudate 303 (e.g., said fibrous plasticized extrudate) onto an assembling stage 390 to form a supporting three dimensional structural framework of one or more fibers 305 defined by the motion of said stage 390, and (c) adding or attaching a controlled amount of drug-containing matter 320 to said supporting fiber structural framework 305 (e.g., said 3D-printed or 3D-micro-patterned supporting fiber structural framework 305).

FIG. 3 further presents a non-limiting method of manufacturing pharmaceutical solid dosage forms comprising the steps of (a) extruding a plasticized matrix 302 comprising at least a first pharmaceutical excipient 301 through an exit port 386 of an extrusion channel 385 by application of mechanical work to form plasticized extrudate 303, (b) depositing (e.g., patterning, printing, etc.) said plasticized extrudate 303 onto an assembling stage 390 to form a supporting solid 300, said supporting solid 300 having an internal three dimensional support structure 305 defined by the motion of said stage 390, and (c) adding or attaching drug-containing matter 320 to said support structure 305 (e.g., said 3D-printed or 3D-micro-patterned support structure 305).

In some embodiments moreover, plasticized extrudate (e.g., deposited plasticized extrudate, fibrous plasticized extrudate, deposited fibrous plasticized extrudate, etc.), three dimensional structural framework of one or more fibers (e.g., deposited three dimensional structural framework of one or more fibers), supporting three dimensional structural framework of one or more fibers (e.g., deposited supporting three dimensional structural framework of one or more fibers), and so on may further be solidified.

FIG. 3 also presents a non-limiting example of a method of manufacturing pharmaceutical solid dosage forms herein that includes some of these embodiments. The method comprises the steps of (a) extruding a plasticized matrix 302 comprising at least a first pharmaceutical excipient 301 through an exit port 386 of an extrusion channel 385 by application of mechanical work to form fibrous plasticized extrudate 303, (b) depositing (e.g., patterning, printing, etc.) said fibrous plasticized extrudate 303 onto an assembling stage 390 (e.g., depositing said fibrous plasticized extrudate 303 onto an assembling stage 390 along apath defined by the motion of said stage 390), (c) solidifying said fibrous plasticized extrudate 303 to form a supporting fibrous structural framework 305 defined by the motion of said stage 390, and (d) adding or attaching a controlled amount of drug-containing matter 320 to said supporting fibrous structural framework 305 (e.g., said 3D-printed or 3D-micro-patterned supporting fiber structural framework, etc.).

FIG. 3 further presents a non-limiting method of manufacturing pharmaceutical solid dosage forms comprising the steps of (a) extruding a plasticized matrix 302 comprising at least a first pharmaceutical excipient 301 through an exit port 386 of an extrusion channel 385 by application of mechanical work to form plasticized extrudate 303, (b) depositing (e.g., patterning, printing, etc.) said plasticized extrudate 303 onto an assembling stage 390 (e.g., depositing said plasticized extrudate 303 onto an assembling stage 390 along a path defined by the motion of said stage 390), (c) solidifying plasticized extrudate 303 to form a supporting solid 300 having an internal three dimensional support structure 305 defined by the motion of said stage 390, and (d) adding or attaching drug-containing matter 320 to said support structure 305 (e.g., said 3D-printed or 3D-micro-patterned support structure, etc.).

FIG. 3 also presents another non-limiting example of a method of manufacturing pharmaceutical solid dosage forms herein. The method comprises the steps of (a) extruding aplasticized matrix 302 comprising at least a first pharmaceutical excipient 301 through an exit port 386 of an extrusion channel 385 by application of mechanical work to form fibrous plasticized extrudate 303, (b) depositing (e.g., patterning, printing, etc.) said fibrous plasticized extrudate 303 onto an assembling stage 390 (e.g., depositing said fibrous plasticized extrudate 303 onto an assembling stage 390 along a path defined by the motion of said stage 390), (c) solidifying said fibrous plasticized extrudate 303 to form a supporting fibrous structural framework 305 defined by the motion of said stage 390, (d) filling (e.g., extruding, squeezing, transporting, etc.) a controlled amount of a drug-containing plasticized matrix 322 into one or more free spaces 330 between fibers or fiber segments of said supporting fiber structural framework 305 (e.g., said 3D-printed or 3D-micro-patterned supporting fiber structural framework), and (e) solidifying said drug-containing plasticized matrix 322 to form drug-containing solid 323 added or attached to said supporting three-dimensional fiber structural framework 305.

FIG. 3 further presents a non-limiting method of manufacturing pharmaceutical solid dosage forms comprising the steps of (a) extruding a plasticized matrix 302 comprising at least a first pharmaceutical excipient 301 through an exit port 386 of an extrusion channel 385 by application of mechanical work to form plasticized extrudate 303, (b) depositing (e.g., patterning, printing, etc.) said plasticized extrudate 303 onto an assembling stage 390 (e.g., depositing said plasticized extrudate 303 onto an assembling stage 390 along a path defined by the motion of said stage 390), (c) solidifying plasticized extrudate 303 to form a supporting solid 300 having an internal three dimensional support structure 305 defined by the motion of said stage 390, (d) filling a controlled amount of a drug-containing plasticized matrix 322 into one or more free spaces 330 (e.g., one or more free spaces 330 of said 3D-printed or 3D-micro-patterned supporting solid 300), and (e) solidifying said drug-containing plasticized matrix 322 to form drug-containing solid 323 attached to said internal structure 305.

In some embodiments, moreover, the step of “adding or attaching a controlled amount of drug-containing matter to a supporting fibrous structural framework (or to a support structure)” may comprise “forming a drug-containing plasticized matrix comprising at least one drug and at least one second pharmaceutical excipient, said drug-containing plasticized matrix disposed in an extrusion channel”, “extruding said drug-containing plasticized matrix through said extrusion channel by application of mechanical work”, “filling a controlled amount of a drug-containing plasticized matrix into one or more fiber-free spaces (or into one or more free spaces)”, and “solidifying said drug-containing plasticized matrix to form drug-containing solid added or attached to said three-dimensional fiber structural framework (or to said support structure)”.

It may be noted that a drug plasticized matrix may generally be formed outside an extruder channel and then filled into said extruder channel, and/or it may be formed right in the extruder channel. The term “forming a drug-containing plasticized matrix comprising at least one drug and at least one second pharmaceutical excipient, said drug-containing plasticized matrix disposed in an extrusion channel” is understood to include at least both of these options.

FIG. 3 also presents a non-limiting example of a method of manufacturing pharmaceutical solid dosage forms herein that includes some of the above embodiments. The method comprises the steps of (a) preparing a supporting three-dimensional structural framework of one or more fibers 305, (b) forming a drug-containing plasticized matrix 321 comprising at least one drug 325 and at least one second pharmaceutical excipient 326, said drug-containing plasticized matrix 321 disposed in an extrusion channel 387, (c) extruding said drug-containing plasticized matrix 321 through said extrusion channel 387 by application of mechanical work, (d) filling (e.g., extruding, squeezing, transporting, etc.) a controlled amount of said extruded drug-containing plasticized matrix 322 into one or more free spaces 330 between fibers or fiber segments of said supporting fiber structural framework 305, and (e) solidifying said drug-containing plasticized matrix 322 to form drug-containing solid 323 added or attached to said supporting fiber structural framework 305.

FIG. 3 also presents another non-limiting example of a method of manufacturing pharmaceutical solid dosage forms herein. The method comprises the steps of (a) preparing a supporting solid 300 comprising an exterior or outer surface 306, an internal, three-dimensional solid support structure 205 contiguous with and terminating at said outer surface 306, and one or more internal, open free spaces 330, (b) forming a drug-containing plasticized matrix 321 comprising at least one drug 325 and at least one second pharmaceutical excipient 326, said drug-containing plasticized matrix 321 disposed in an extrusion channel 387, (c) extruding said drug-containing plasticized matrix 321 through said extrusion channel 387 by application of mechanical work (d) filling (e.g., extruding, squeezing, transporting, etc.) a controlled amount of said extruded drug-containing plasticized matrix 322 into one or more free spaces 330 of said supporting solid 300, and (e) solidifying said drug-containing plasticized matrix 322 to form drug-containing solid 323 attached to said support structure 305.

FIG. 3 further presents a non-limiting example of a method of manufacturing pharmaceutical solid dosage forms herein comprising (a) extruding a plasticized matrix 302 comprising at least a first pharmaceutical excipient 301 through an exit port 386 of an extrusion channel 385 by application of mechanical work to form fibrous plasticized extrudate 303, (b) depositing (e.g., patterning, printing, etc.) said fibrous plasticized extrudate 303 onto an assembling stage 390 (e.g., depositing said fibrous plasticized extrudate 303 onto an assembling stage 390 along a path defined by the motion of said stage 390), (c) solidifying said fibrous plasticized extrudate 303 to form a supporting fibrous structural framework 305 defined by the motion of said stage 390, (d) forming a drug-containing plasticized matrix 321 comprising at least one drug 325 and at least one second pharmaceutical excipient 326, said drug-containing plasticized matrix 321 disposed in an extrusion channel 387, (e) extruding said drug-containing plasticized matrix 321 through said extrusion channel 387 by application of mechanical work (f) filling (e.g., extruding, squeezing, transporting, etc.) a controlled amount of said extruded drug-containing plasticized matrix 322 into one or more free spaces 330 between fibers or fiber segments of said supporting fiber structural framework 305 (e.g., said 3D-printed or 3D-micro-patterned supporting fiber structural framework), and (g) solidifying said drug-containing plasticized matrix 322 to form drug-containing solid attached to said supporting fiber structural framework.

FIG. 3 also presents another non-limiting example of a method of manufacturing pharmaceutical solid dosage forms herein comprising (a) extruding a plasticized matrix 302 comprising at least a first pharmaceutical excipient 301 through an exit port 386 of an extrusion channel 385 by application of mechanical work to form plasticized extrudate 303, (b) depositing (e.g., patterning, printing, etc.) said plasticized extrudate 303 onto an assembling stage 390 (e.g., depositing said plasticized extrudate 303 onto an assembling stage 390 along a path defined by the motion of said stage 390), (c) solidifying plasticized extrudate 303 to form a supporting solid 300 having an internal three dimensional support structure 305 defined by the motion of said stage 390, (d) forming a drug-containing plasticized matrix 321 comprising at least one drug 325 and at least one second pharmaceutical excipient 326, said drug-containing plasticized matrix 321 disposed in an extrusion channel 387, (e) extruding said drug-containing plasticized matrix 321 through said extrusion channel 387 by application of mechanical work, (f) filling (e.g., extruding, squeezing, transporting, etc.) a controlled amount of said extruded drug-containing plasticized matrix 322 into one or more free spaces 330 (e.g., one or more free spaces 330 of said 3D-printed or 3D-micro-patterned supporting solid 300), and (g) solidifying said drug-containing plasticized matrix 322 to form drug-containing solid 323 attached to said support structure 305.

In some embodiments, the method of manufacturing pharmaceutical solid dosage forms herein may further comprise the step of coating a three-dimensional structural framework of one or more fibers or apart (e.g., a fraction) thereof with a coating. Similarly, the method of manufacturing pharmaceutical solid dosage forms herein may further comprise the step of coating a supporting three-dimensional structural framework of one or more fibers or a part (e.g., a fraction) thereof with a coating.

Moreover, in some embodiments, the method of manufacturing pharmaceutical solid dosage forms herein may further comprise the step of coating a support structure of a supporting solid or a part (e.g., a fraction) thereof with a coating.

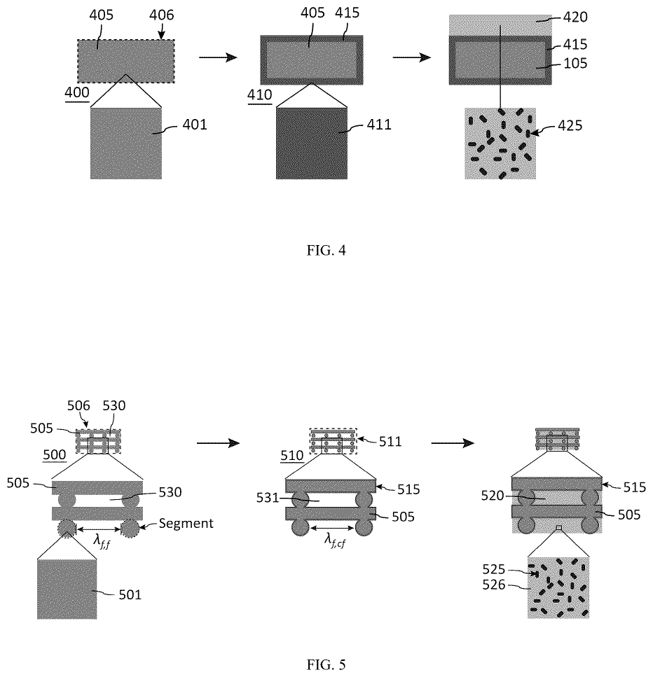

FIG. 4 presents a non-limiting method of manufacturing pharmaceutical solid dosage forms according to the invention herein that include some of the above embodiments. The method comprises the steps of (a) preparing (e.g., manufacturing) a supporting solid 400 comprising an outer surface 406 and an internal, three dimensional support structure 405 contiguous with and terminating at said outer surface 406, (b) coating said internal support structure 405 with a coating 415 to form a coated support structure 405,415, and (c) adding or attaching drug-containing matter 420 to said coated support structure 405,415. It may be noted that the term “adding or attaching drug-containing matter 420 to said coated support structure 405,415” includes, but is not limited to “adding or attaching drug-containing matter 420 to a surface or a deposition surface of said coated support structure 405,415”, and so on.

Generally, a support structure 405 may comprise at least a first excipient 401. Similarly, a drug-containing matter 420 may comprise at least a drug 426. Additionally, a coating 415 may generally comprise at least a third pharmaceutical excipient 411. Said third pharmaceutical excipient 411 may generally comprise any material suitable for oral ingestion by a human or animal subject. Preferably, moreover, said third pharmaceutical excipient 411 may be solid at room temperature. Said third pharmaceutical excipient 411 is also referred to herein as “third excipient”.

FIG. 5 presents another non-limiting method of manufacturing pharmaceutical solid dosage forms herein that comprises the steps of (a) preparing (e.g., manufacturing) a supporting solid 500 comprising an outer surface 506 and an internal, three dimensional structural framework of one or more fibers 505, said fibrous structural framework 505 contiguous with and terminating at said outer surface 506, (b) coating said fibrous structural framework 505 with a coating 515 to form a coated three-dimensional fiber structural framework 505,515, and (c) adding or attaching drug-containing matter 520 to said coated three-dimensional fiber structural framework 505,515. It may be noted that the term “adding or attaching drug-containing matter 520 to said coated three-dimensional fiber structural framework 505,515” includes, but is not limited to “adding or attaching drug-containing matter 520 to a surface or a deposition surface of said coated three-dimensional fiber structural framework 505,515”, and so on.

FIG. 5 also presents a non-limiting method of manufacturing pharmaceutical solid dosage forms herein comprising the steps of (a) preparing (e.g., manufacturing) a supporting three-dimensional structural framework of one or more fibers 505, (b) coating said supporting three-dimensional fibrous structural framework 505 with a coating 515 to form a coated three-dimensional fiber structural framework 505,515, and (c) adding or attaching a controlled amount of drug-containing matter 520 to said coated supporting fiber structural framework 505,515. It may be noted that the term “adding or attaching a controlled amount of drug-containing matter 520 to said coated supporting fiber structural framework 505,515” includes, but is not limited to “adding or attaching a controlled amount of drug-containing matter 520 to a surface or a deposition surface of said coated supporting fiber structural framework 505,515”, and so on.

Generally, moreover, one or more coated fibers or one or more coated fiber segments in a coated supporting three dimensional fibrous structural framework 505,515 may be spaced apart from adjoining coated fibers or coated fiber segments by coated-fiber-free spacings, eff, defining one or more coated-fiber-free spaces 531 within an outer volume of said coated fibrous structural framework 505,515. In the invention herein, an “outer volume of a coated fibrous structural framework” may generally be understood as “envelope” of said “coated fibrous structural framework 505,515”.

Generally, furthermore, one or more coated-fiber-free spaces 531 (e.g., one or more free spaces of a coated supporting solid) may be substantially open to an exterior surface 511 of a coated fibrous structural framework 510 (e.g., an exterior surface of a coated supporting solid). In other words, one or more coated-fiber-free spaces 531 (e.g., one or more free spaces of a coated supporting solid) may generally have at least one open end contiguous with and terminating at an exterior or outer surface 511 of a coated fibrous structural framework 510 (e.g., an exterior or outer surface of a coated supporting solid).

In some embodiments, a coated-fiber-free space 531 (or a free space of a coated supporting solid) that is open may be filled with drug-containing matter 520 upon adding or attaching a controlled amount of drug-containing matter 520 to a coated supporting fiber structural framework 505,515 (or to a coated support structure).

Thus, by way of example but not by way of limitation, in some embodiments the step of “adding or attaching a controlled amount of drug-containing matter to a coated supporting fiber structural framework (or to a coated support structure)” may comprise “filling a controlled amount of a drug-containing plasticized matrix into one or more coated-fiber-free spaces (or into one or more free spaces of a coated supporting solid)”, and “solidifying said drug-containing plasticized matrix to form drug-containing solid added or attached to said coated three-dimensional fiber structural framework (or to said coated support structure)”.

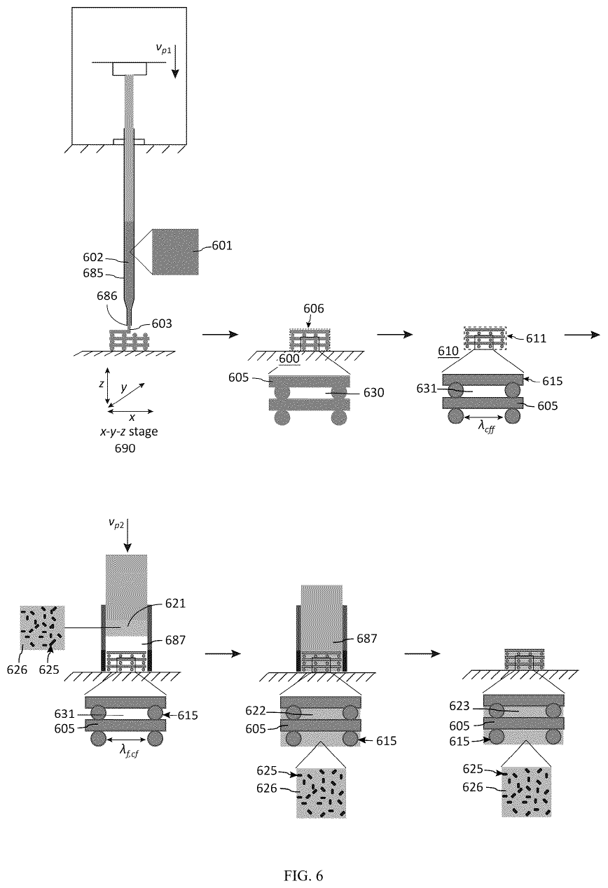

FIG. 6 presents a non-limiting example of a method of manufacturing pharmaceutical solid dosage forms herein that includes some of the above embodiments. The method comprises the steps of (a) preparing a supporting three-dimensional structural framework of one or more fibers 605, (b) coating said supporting three-dimensional fibrous structural framework 605 with a coating 615 to form a coated supporting three-dimensional fiber structural framework 605,615, (c) filling (e.g., extruding, squeezing, transporting, etc.) a controlled amount of a drug-containing plasticized matrix 622 into one or more free spaces 631 between coated fibers or coated fiber segments of said coated supporting fiber structural framework 605,615, and (d) solidifying said drug-containing plasticized matrix 622 to form drug-containing solid 623 added or attached to said coated supporting three-dimensional fiber structural framework 605,615.

FIG. 6 also presents another non-limiting example of a method of manufacturing pharmaceutical solid dosage forms according to this invention. The method comprises the steps of (a) preparing a supporting solid 600 comprising an exterior or outer surface 606, an internal, three-dimensional solid support structure 605 contiguous with and terminating at said outer surface 606, and one or more internal, open free spaces 630, (b) coating said internal support structure 605 with a coating 615 to form a coated supporting solid 610, (c) filling (e.g., extruding, squeezing, transporting, etc.) a controlled amount of a drug-containing plasticized matrix 622 into one or more internal, open free spaces 631 of said coated supporting solid 610, and (d) solidifying said drug-containing plasticized matrix 622 to form drug-containing solid 623 added or attached to said coated support structure 605,615.

FIG. 6 also presents a non-limiting method of manufacturing pharmaceutical solid dosage forms herein comprising the steps of (a) extruding a plasticized matrix 602 comprising at least a first pharmaceutical excipient 601 through an exit port 686 of an extrusion channel 685 by application of mechanical work to form plasticized extrudate 603, (b) depositing (e.g., patterning, printing, etc.) said plasticized extrudate 603 onto an assembling stage 690 to form a supporting three dimensional structural framework of one or more fibers 605 defined by the motion of said stage 690, (c) coating said supporting three-dimensional fibrous structural framework 605 with a coating 615, and (d) adding or attaching a controlled amount of drug-containing matter 620 to said coated supporting fiber structural framework 605,615 (e.g., said coated, 3D-printed or 3D-micro-patterned supporting fiber structural framework 605,615).

FIG. 6 further presents a non-limiting method of manufacturing pharmaceutical solid dosage forms comprising the steps of (a) extruding a plasticized matrix 602 comprising at least a first pharmaceutical excipient 601 through an exit port 686 of an extrusion channel 685 by application of mechanical work to form plasticized extrudate 603, (b) depositing (e.g., patterning, printing, etc.) said plasticized extrudate 603 onto an assembling stage 690 to form a supporting solid 600, said supporting solid 600 having an internal three dimensional support structure 605 defined by the motion of said stage 690, (c) coating said internal support structure 605 with a coating 615, and (d) adding or attaching drug-containing matter 620 to said coated support structure 605,615 (e.g., said coated, 3D-printed or 3D-micro-patterned support structure 605,615).

FIG. 6 also presents another non-limiting method of manufacturing pharmaceutical solid dosage forms herein comprising (a) extruding a plasticized matrix 602 comprising at least a first pharmaceutical excipient 601 through an exit port 686 of an extrusion channel 685 by application of mechanical work to form fibrous plasticized extrudate 603, (b) depositing (e.g., patterning, printing, etc.) said fibrous plasticized extrudate 603 onto an assembling stage 690 (e.g., depositing said fibrous plasticized extrudate 603 onto an assembling stage 690 along a path defined by the motion of said stage 690), (c) solidifying said fibrous plasticized extrudate 603 to form a supporting fibrous structural framework 605 defined by the motion of said stage 690, (d) coating said supporting three-dimensional fibrous structural framework 605 with a coating 615, and (e) adding or attaching a controlled amount of drug-containing matter 620 to said coated supporting fibrous structural framework 605,615 (e.g., said coated, 3D-printed or 3D-micro-patterned supporting fiber structural framework 605,615, etc.).

FIG. 6 further presents a non-limiting method of manufacturing pharmaceutical solid dosage forms comprising the steps of (a) extruding a plasticized matrix 602 comprising at least a first pharmaceutical excipient 601 through an exit port 686 of an extrusion channel 685 by application of mechanical work to form plasticized extrudate 603, (b) depositing (e.g., patterning, printing, etc.) said plasticized extrudate 603 onto an assembling stage 690 (e.g., depositing said plasticized extrudate 603 onto an assembling stage 690 along a path defined by the motion of said stage 690), (c) solidifying plasticized extrudate 603 to form a supporting solid 600 having an internal three dimensional support structure 605 defined by the motion of said stage 690, (d) coating said internal support structure 605 with a coating 615, and (e) adding or attaching drug-containing matter 620 to said coated support structure 605,615 (e.g., said coated, 3D-printed or 3D-micro-patterned support structure 605,615, etc.).

FIG. 6 also presents another non-limiting example of a method of manufacturing pharmaceutical solid dosage forms herein. The method comprises the steps of (a) extruding a plasticized matrix 602 comprising at least a first pharmaceutical excipient 601 through an exit port 686 of an extrusion channel 685 by application of mechanical work to form plasticized extrudate 603, (b) depositing (e.g., patterning, printing, etc.) said fibrous plasticized extrudate 603 onto an assembling stage 690 (e.g., depositing said fibrous plasticized extrudate 603 onto an assembling stage 690 along a path defined by the motion of said stage 690), (c) solidifying said fibrous plasticized extrudate 603 to form a supporting fibrous structural framework 605 defined by the motion of said stage 690, (d) coating said supporting three-dimensional fibrous structural framework 605 with a coating 615, (e) filling (e.g., extruding, squeezing, transporting, etc.) a controlled amount of a drug-containing plasticized matrix 622 into one or more free spaces 631 between coated fibers or coated fiber segments of said coated supporting fiber structural framework 605,615 (e.g., said coated, 3D-printed or 3D-micro-patterned supporting fiber structural framework 605,615), and (f) solidifying said drug-containing plasticized matrix 622 to form drug-containing solid 623 added or attached to said coated supporting three-dimensional fiber structural framework 605,615.

FIG. 6 further presents a non-limiting method of manufacturing pharmaceutical solid dosage forms comprising the steps of (a) extruding a plasticized matrix 602 comprising at least a first pharmaceutical excipient 601 through an exit port 686 of an extrusion channel 685 by application of mechanical work to form plasticized extrudate 603, (b) depositing (e.g., patterning, printing, etc.) said plasticized extrudate 603 onto an assembling stage 690 (e.g., depositing said plasticized extrudate 603 onto an assembling stage 690 along a path defined by the motion of said stage 690), (c) solidifying plasticized extrudate 603 to form a supporting solid 600 having an internal three dimensional support structure 605 defined by the motion of said stage 690, (d) coating said internal support structure 605 with a coating 615, (e) filling a controlled amount of a drug-containing plasticized matrix 622 into one or more free spaces 631 of said coated supporting solid 610 (e.g., one or more free spaces of said coated, 3D-printed or 3D-micro-patterned supporting solid 610), and (f) solidifying said drug-containing plasticized matrix 622 to form drug-containing solid 623 attached to said coated internal structure 605,615.

FIG. 6 also presents a non-limiting example of a method of manufacturing pharmaceutical solid dosage forms herein that includes some of the above embodiments. The method comprises the steps of (a) preparing a supporting three-dimensional structural framework of one or more fibers 605, (b) coating said supporting three-dimensional fibrous structural framework 605 with a coating 615, (c) forming a drug-containing plasticized matrix 621 comprising at least one drug 625 and at least one second pharmaceutical excipient 625, said drug-containing plasticized matrix 621 disposed in an extrusion channel 687, (d) extruding said drug-containing plasticized matrix 621 through said extrusion channel by application of mechanical work, (e) filling (e.g., extruding, squeezing, transporting, etc.) a controlled amount of said extruded drug-containing plasticized matrix 622 into one or more free spaces 631 between coated fibers or coated fiber segments of said coated supporting fiber structural framework 605,615, and (f) solidifying said drug-containing plasticized matrix 622 to form drug-containing solid 623 attached to said coated supporting fiber structural framework 605,615.

FIG. 6 also presents another non-limiting example of a method of manufacturing pharmaceutical solid dosage forms herein. The method comprises the steps of (a) preparing a supporting solid 600 comprising an exterior or outer surface 606, an internal, three-dimensional solid support structure 605 contiguous with and terminating at said outer surface 606, and one or more internal, open free spaces 630, (b) coating said internal support structure 605 with a coating 615 to form a coated supporting solid 610, (c) forming a drug-containing plasticized matrix 621 comprising at least one drug 625 and at least one second pharmaceutical excipient 626, said drug-containing plasticized matrix 625 disposed in an extrusion channel 687, (d) extruding said drug-containing plasticized matrix 621 through said extrusion channel 687 by application of mechanical work (e) filling (e.g., extruding, squeezing, transporting, etc.) a controlled amount of said extruded drug-containing plasticized matrix 622 into one or more free spaces 631 of said coated supporting solid 610, and (e) solidifying said drug-containing plasticized matrix 622 to form drug-containing solid 623 attached to said coated support structure 605,615.

FIG. 6 further presents a non-limiting example of a method of manufacturing pharmaceutical solid dosage forms herein comprising (a) extruding a plasticized matrix 602 comprising at least a first pharmaceutical excipient 601 through an exit port 686 of an extrusion channel 685 by application of mechanical work to form fibrous plasticized extrudate 603, (b) depositing (e.g., patterning, printing, etc.) said fibrous plasticized extrudate 603 onto an assembling stage 690 (e.g., depositing said fibrous plasticized extrudate 603 onto an assembling stage 690 along a path defined by the motion of said stage 690), (c) solidifying said fibrous plasticized extrudate 603 to form a supporting fibrous structural framework 605 defined by the motion of said stage 690, (d) coating said supporting three-dimensional fibrous structural framework 605 with a coating 615, (e) forming a drug-containing plasticized matrix 621 comprising at least one drug 625 and at least one second pharmaceutical excipient 626, said drug-containing plasticized matrix 621 disposed in an extrusion channel 687, (f) extruding said drug-containing plasticized matrix 621 through said extrusion channel 687 by application of mechanical work (g) filling (e.g., extruding, squeezing, transporting, etc.) a controlled amount of said extruded drug-containing plasticized matrix 622 into one or more free spaces 631 between coated fibers or coated fiber segments of said coated supporting fiber structural framework 605,615 (e.g., said coated, 3D-printed or 3D-micro-patterned supporting fiber structural framework 605,615), and (f) solidifying said drug-containing plasticized matrix 622 to form drug-containing solid 623 attached to said coated supporting fiber structural framework 605,615.

FIG. 6 also presents another non-limiting example of a method of manufacturing pharmaceutical solid dosage forms herein comprising (a) extruding a plasticized matrix 602 comprising at least a first pharmaceutical excipient 601 through an exit port 686 of an extrusion channel 685 by application of mechanical work to form plasticized extrudate 603, (b) depositing (e.g., patterning, printing, etc.) said plasticized extrudate 603 onto an assembling stage 690 (e.g., depositing said plasticized extrudate 603 onto an assembling stage 690 along a path defined by the motion of said stage 690), (c) solidifying plasticized extrudate 603 to form a supporting solid 600 having an internal three dimensional support structure 605 defined by the motion of said stage 690, (d) coating said internal support structure 605 with a coating 615 to form a coated supporting solid 610, (e) forming a drug-containing plasticized matrix 621 comprising at least one drug 625 and at least one second pharmaceutical excipient 626, said drug-containing plasticized matrix 621 disposed in an extrusion channel 687, (f) extruding said drug-containing plasticized matrix 621 through said extrusion channel 687 by application of mechanical work, (g) filling (e.g., extruding, squeezing, transporting, etc.) a controlled amount of said extruded drug-containing plasticized matrix 622 into one or more free spaces 631 of said coated supporting solid 610 (e.g., one or more free spaces 631 of said coated, 3D-printed or 3D-micro-patterned supporting solid 610), and (f) solidifying said drug-containing plasticized matrix 622 to form drug-containing solid 623 attached to said coated support structure 605,615 (e.g., solidifying said drug-containing plasticized matrix 622 within said one or more free spaces 631 to form drug-containing solid 623 attached to said coated support structure 605,615).

In some embodiments, moreover, a method of manufacturing pharmaceutical solid dosage forms according to this invention may comprise or further comprise the step of pushing a needle or pin into or through drug-containing matter to form one or more channels within said drug-containing matter. This includes, but is not limited to a method of manufacturing pharmaceutical solid dosage forms further comprising the step of pushing a needle or pin into or through drug-containing plasticized matrix to form one or more channels within said drug-containing plasticized matrix.

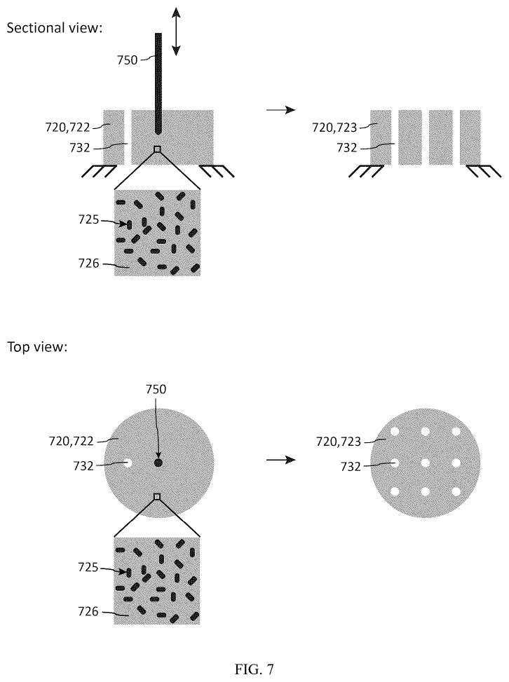

FIG. 7 presents another non-limiting example of a method of manufacturing pharmaceutical solid dosage forms according to the invention herein. The method comprises the step of pushing a needle or pin 750 into or through drug-containing matter 720 to form one or more channels 732 within said drug-containing matter 720. Generally, said drug-containing matter may comprise at least a drug 725 and at least second pharmaceutical excipient 726.

FIG. 7 also presents another non-limiting example of a method of manufacturing pharmaceutical solid dosage forms according to the invention herein. The method comprises the steps of (a) forming a drug-containing plasticized matrix 722 comprising at least a drug 725 and at least a second pharmaceutical excipient 726, (b) pushing a needle or pin 750 into or through said drug-containing plasticized matrix 722 to form one or more channels 732 within said drug-containing plasticized matrix 722, and (c) solidifying said drug-containing plasticized matrix 722 to form a drug-containing solid 723.

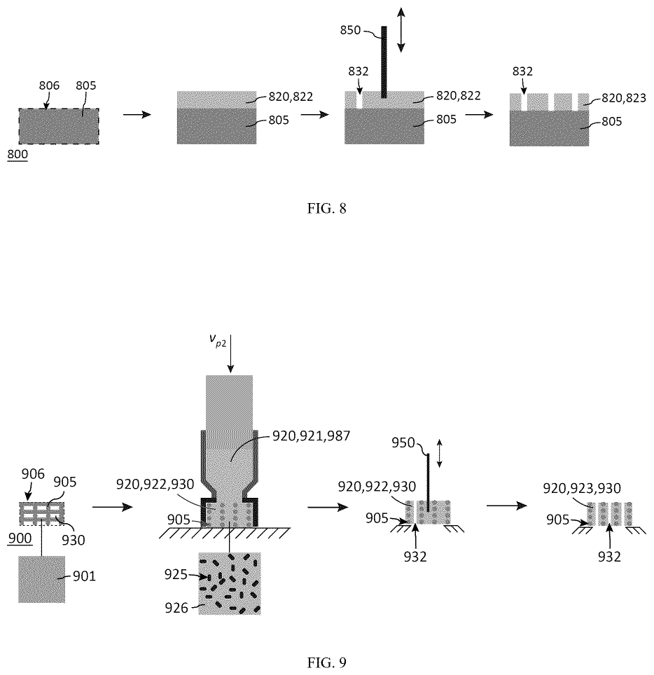

FIG. 8 presents another non-limiting example of a method of manufacturing pharmaceutical solid dosage forms according to the invention herein. The method comprises the steps of (a) preparing a supporting solid 800 comprising an outer surface 806 and an internal, three dimensional structure 805 contiguous with and terminating at said outer surface 806, (b) adding or attaching drug-containing matter 820 to said internal structure 805, and (c) pushing a needle or pin 850 into or through said drug-containing matter 820 to form one or more channels 832 within said drug-containing matter 820.

FIG. 8 also presents another non-limiting example of a method of manufacturing pharmaceutical solid dosage forms according to the invention herein. The method comprises the steps of (a) preparing a supporting solid 800 comprising an outer surface 806 and an internal, three dimensional structure 805 contiguous with and terminating at said outer surface 806, (b) adding or attaching drug-containing plasticized matrix 822 to said internal structure 805, (c) pushing a needle or pin 850 into or through said drug-containing plasticized matrix 822 to form one or more channels 832 within said drug-containing plasticized matrix 822, and (d) solidifying said drug-containing plasticized matrix 822 to form a drug-containing solid 823 added or attached to said support structure 805.

FIG. 9 presents another non-limiting example of a method of manufacturing pharmaceutical solid dosage forms according to the invention herein. The method comprises the steps of: (a) preparing a supporting three-dimensional structural framework of one or more fibers 905 comprising at least a first excipient 901, (b) adding or attaching drug-containing matter 920 to said three-dimensional fiber structural framework 905, and (c) pushing a needle or pin 950 into or through said drug-containing matter 920 to form one or more channels 932 within said drug-containing matter 920.

FIG. 9 further presents a non-limiting example of a method of manufacturing pharmaceutical solid dosage forms herein comprising the steps of: (a) preparing a supporting three-dimensional structural framework of one or more fibers 905, (b) adding or attaching a controlled amount of drug-containing plasticized matrix 922 to said three-dimensional fiber structural framework 905, (c) pushing a needle or pin 950 into or through said drug-containing plasticized matrix 922 to form one or more channels 932 within said drug-containing plasticized matrix 922, and (d) solidifying said drug-containing plasticized matrix 922 to form a drug-containing solid 923 added or attached to said supporting fibrous structural framework 905.

FIG. 9 further presents a non-limiting example of a method of manufacturing pharmaceutical solid dosage forms herein comprising the steps of: (a) preparing a supporting three-dimensional structural framework of one or more fibers 905, (b) adding or attaching a controlled amount of drug-containing plasticized matrix 922 to said three-dimensional fiber structural framework 905, (c) pushing a needle or pin 950 into or through said drug-containing plasticized matrix 922 to form one or more channels 932 within said drug-containing plasticized matrix 922, and (d) solidifying said drug-containing plasticized matrix 922 to form a drug-containing solid 923 added or attached to said supporting fibrous structural framework 905.

FIG. 9 further presents a non-limiting example of a method of manufacturing pharmaceutical solid dosage forms herein comprising the steps of: (a) preparing a supporting three-dimensional structural framework of one or more fibers 905, (b) forming a drug-containing plasticized matrix 921 comprising at least one drug 925 and at least one second pharmaceutical excipient 926, said drug-containing plasticized matrix 921 disposed in an extrusion channel 987, (c) extruding said drug-containing plasticized matrix 921 through said extrusion channel 987 by application of mechanical work, (d) filling (e.g., extruding, squeezing, transporting, etc.) a controlled amount of said extruded drug-containing plasticized matrix 922 into one or more free spaces 930 between fibers or fiber segments of said supporting fiber structural framework 905, (e) pushing a needle or pin 950 into or through said drug-containing plasticized matrix 922 to form one or more channels 932 within said drug-containing plasticized matrix 922, and (f) solidifying said drug-containing plasticized matrix 922 to form a drug-containing solid 923 added or attached to said supporting fibrous structural framework 905.

FIG. 9 also presents another non-limiting example of a method of manufacturing pharmaceutical solid dosage forms herein comprising (a) preparing (e.g., producing, manufacturing, 3D-printing, 3D-patterning, printing, patterning, etc.) a supporting solid 900 having an outer surface 906, an internal three dimensional support structure 905 contiguous with an terminating at said outer surface 906, and one or more internal, open free spaces 930, (b) forming a drug-containing plasticized matrix 921 comprising at least one drug 925 and at least one second pharmaceutical excipient 926, said drug-containing plasticized matrix 921 disposed in an extrusion channel 987, (c) extruding said drug-containing plasticized matrix 921 through said extrusion channel 987 by application of mechanical work, (d) filling (e.g., extruding, squeezing, transporting, etc.) a controlled amount of said extruded drug-containing plasticized matrix 922 into one or more free spaces 930 (e.g., to add (e.g., combine, join, connect, attach, bond, immovably attach, etc.) said drug-containing plasticized matrix 922 to said three-dimensional support structure 905), (e) pushing a needle or pin 950 into or through said drug-containing plasticized matrix 922 within said one or more free spaces 930 to form one or more channels 932 within said drug-containing plasticized matrix 922 (e.g., in the drug-containing plasticized matrix 922 that partially or substantially fills one or more free spaces 930 in said supporting solid 900, in the drug-containing plasticized matrix 922 added to said support structure 905, etc.), and (f) solidifying said drug-containing plasticized matrix 922 to form a drug-containing solid 923 added or attached to said support structure 905.

FIG. 9 also presents another non-limiting example of a method of manufacturing pharmaceutical solid dosage forms according to this invention. The method may comprise the steps of (a) preparing (e.g., producing, manufacturing, 3D-printing, 3D-patterning, printing, patterning, etc.) a supporting solid 900 having an outer surface 906, an internal, three dimensional structural framework of criss-crossed stacked layers of one or more fibers 905 contiguous with and terminating at said outer surface 906 and comprising at least a first pharmaceutical excipient 901, and one or more internal, open free spaces 930, (b) forming a drug-containing plasticized matrix 921 comprising at least a drug 925 and at least a second pharmaceutical excipient 926, (c) filling (e.g., extruding, transporting, squeezing, etc.) said drug-containing plasticized matrix 921,922 into one or more internal, open free spaces 930 of said supporting solid 900 (e.g., to add (e.g., combine, join, connect, attach, bond, immovably attach, etc.) said drug-containing plasticized matrix 922 to said three dimensional structural framework of criss-crossed stacked layers of one or more fibers 905), and (d) pushing a needle or pin 950 into or through drug-containing plasticized matrix 922 within one or more internal, open free spaces 930 of said supporting solid 900 (e.g., into or through one or more free spaces 930 between one or more fibers or fiber segments of said fiber structural framework 905) to form one or more channels 932 in said drug-containing plasticized matrix 922 (e.g., in the drug-containing plasticized matrix 922 that partially or substantially fills one or more free spaces 930 in said supporting solid 900, in the drug-containing plasticized matrix 922 added to said three dimensional structural framework of criss-crossed stacked layers of one or more fibers 905, etc.), and (f) solidifying said drug-containing plasticized matrix 922 to form a drug-containing solid 923 added or attached to said supporting fibrous structural framework 905.

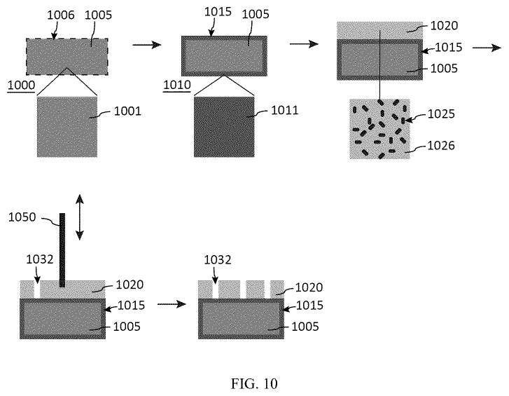

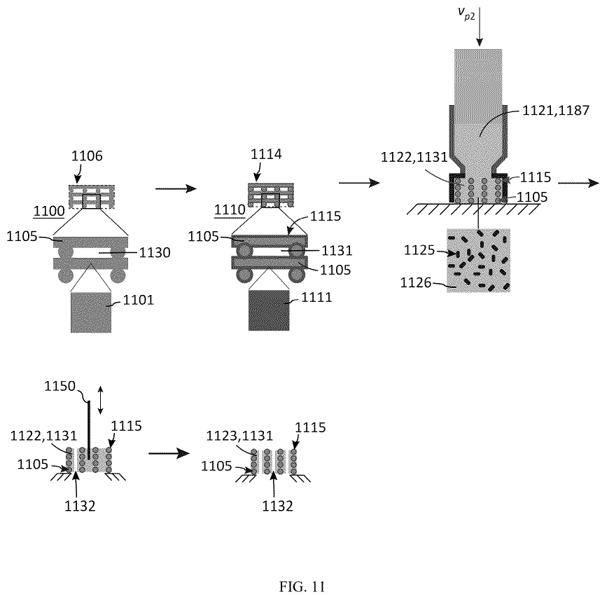

FIG. 10 presents another method of manufacturing pharmaceutical solid dosage forms herein comprising the steps of (a) preparing a supporting solid 1000 comprising an outer surface 1006 and an internal, three dimensional support structure 1005 contiguous with and terminating at said outer surface 1006, said support structure 1005 comprising at least a first excipient 1001, (b) coating said support structure 1005 or a part thereof with a coating 1015 comprising at least a third pharmaceutical excipient 1011, (c) adding or attaching drug-containing matter 1020 comprising at least a drug 1025 and at least a second excipient 1026 to said support structure 1005, and (d) pushing a needle or pin 1050 into or through said drug-containing matter 1020 to form one or more channels 1032 within said drug-containing matter 1020.

FIG. 11 presents a further method of manufacturing pharmaceutical solid dosage forms according to the invention herein. The method comprises the steps of (a) preparing a supporting three-dimensional structural framework of one or more fibers 1105, (b) coating said supporting three-dimensional fiber structural framework 1105 or a part thereof with a coating 1115, (c) adding or attaching drug-containing matter 1120 to said coated three-dimensional fiber structural framework 1105,1115, and (d) pushing a needle or pin 1150 into or through said drug-containing matter 1120 to form one or more channels 1132 within said drug-containing matter 1132.

FIG. 11 also presents another non-limiting example of a method of manufacturing pharmaceutical solid dosage forms according to this invention. The method may comprise the steps of (a) preparing (e.g., manufacturing) a supporting solid 1100 having an outer surface 1106, an internal, three dimensional support structure 1105 contiguous with and terminating at said outer surface 1106, and one or more internal, open support-structure-free spaces 1130, wherein said internal, three dimensional support structure 1105 comprises at least a first pharmaceutical excipient 1101, (b) coating (e.g., substantially encapsulating, substantially coating, substantially surrounding, substantially enclosing, etc.) said three-dimensional support structure 1105 with a coating 1115 comprising at least a second pharmaceutical excipient 1111 to form a coated supporting solid 1110 having an outer surface 1114, a coated, internal support structure 1105,1115 contiguous with and terminating at said outer surface 1114, and one or more internal coated-support-structure-free spaces 1131, (c) forming a drug-containing plasticized matrix 1121 comprising at least a drug 1125 and at least a second pharmaceutical excipient 1126, (d) filling (e.g., extruding, transporting, squeezing, etc.) said drug-containing plasticized matrix 1121,1122 into one or more coated-support-structure-free spaces 1131 of said coated supporting solid 1110, (e) pushing a needle or pin 1150 into or through drug-containing plasticized matrix 1122 within one or more coated-support-structure-free spaces 1131 of said coated supporting solid 1110 to form one or more channels 1132 in said drug-containing plasticized matrix 1122 (e.g., in the drug-containing plasticized matrix 1122 that partially or substantially fills one or more coated-support-structure-free spaces 1131 in said coated supporting solid 1110, in the drug-containing plasticized matrix 1122 attached to said coated support structure 1105,1115, etc.), and (f) solidifying said drug-containing plasticized matrix 1122 to form a drug-containing solid 1123 added or attached to said coated support structure 1105,1115.

In some embodiments, moreover, a supporting solid herein may comprise an exterior or outer surface, an internal, three-dimensional structural framework of criss-crossed stacked layers of one or more fibers, said framework contiguous with and terminating at said outer surface, and one or more internal, open fiber-free spaces. Similarly, in some embodiments, a coated supporting solid herein may comprise an exterior or outer surface, an internal three-dimensional structural framework of criss-crossed stacked layers of one or more coated fibers, said coated framework contiguous with and terminating at said outer surface, and one or more coated-fiber-free spaces.

Additional aspects and embodiments of the method disclosed herein are described throughout this specification. Any more aspects and embodiments that would be obvious to a person of ordinary skill in the art are all within the spirit and scope of this invention.

### Additional Embodiments

In addition to the aspects and embodiments disclosed above, the method of manufacturing pharmaceutical solid dosage forms disclosed herein may comprise the following embodiments.

### (a) Embodiments Related to the Preparation of Supporting Solid

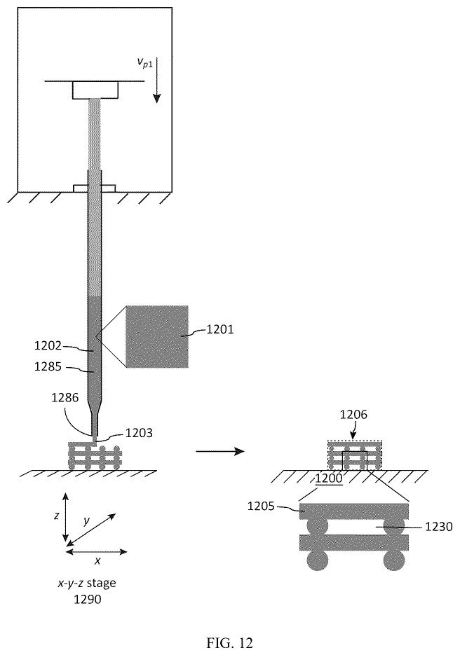

FIG. 12 presents a non-limiting example of a method of manufacturing supporting solids as disclosed herein. A plasticized matrix 1202 comprising at least a first pharmaceutical excipient 1201 may be extruded through an exit port 1286 of an extrusion channel 1285 by application of mechanical work to form plasticized extrudate 1203. Said plasticized extrudate 1203 may then be deposited (e.g., patterned, printed, etc.) onto an assembling stage 1290 (e.g., deposited along a path defined by the motion of said stage 1290) to form a supporting solid 1200 having an internal three dimensional support structure 1205 defined by the motion of said stage 1290. It may be noted, moreover, that generally, a plasticized matrix may be prepared (e.g., produced, formed, etc.) before or during extrusion through an exit port of an extrusion channel. Similarly, a plasticized matrix may, for example, be prepared (e.g., produced, formed, etc.) inside an extrusion channel, and/or it may be prepared outside an extrusion channel and then be filled (e.g., fed, injected, transported, etc.) into said extrusion channel.

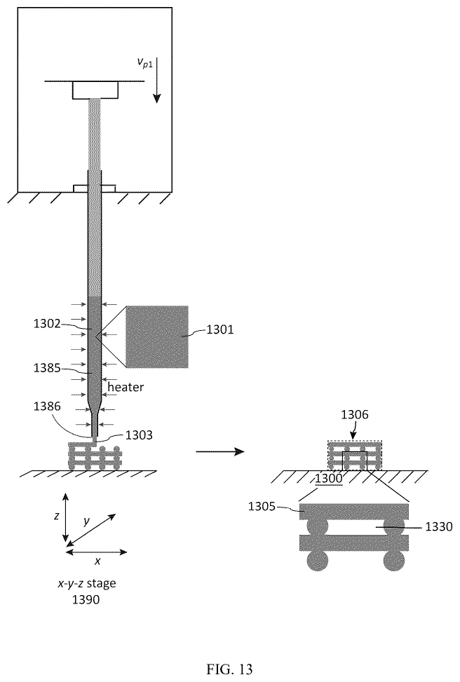

In some embodiments, a plasticized matrix may be prepared by heating a composition comprising at least one first excipient. FIG. 13 presents a non-limiting method of manufacturing supporting solids that includes some of these embodiments. A plasticized matrix 1302 comprising at least a first pharmaceutical excipient 1301 may first be prepared by heating said at least one first excipient 1301. Said plasticized matrix 1302 may be disposed (e.g., placed, located, positioned, filled, etc.) in an extrusion channel 1385. Said plasticized matrix 1302 may then be extruded through an exit port 1386 of said extrusion channel 1385 by application of mechanical work to form plasticized extrudate 1303. Said plasticized extrudate 1303 may then be deposited (e.g., patterned, printed, etc.) onto an assembling stage 1390 (e.g., deposited along a path defined by the motion of said stage 1390) to form a supporting solid 1300 having a three dimensional support structure 1305 defined by the motion of said stage 1390. It may be noted, moreover, that generally, a heated plasticized extrudate 1303 may also be cooled during or after deposition on an assembling stage 1390 to solidify said plasticized extrudate 1303. In the invention herein, the term “cooling a plasticized extrudate” is generally referred to as “reducing the temperature of a plasticized extrudate”.

In some embodiments, however, a desirable pharmaceutical composition may not plasticize adequately upon heating alone, or it may for other reasons not be processable at elevated temperatures. In such (and other) embodiments, plasticized matrix may, for example, be prepared by solvating at least a first excipient with a first solvent.

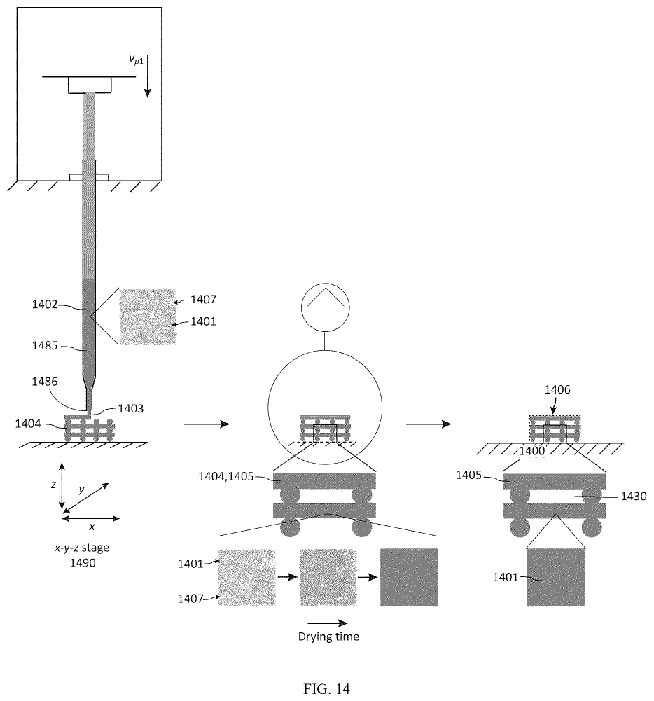

FIG. 14 presents a non-limiting method of manufacturing supporting solids herein that includes some of these embodiments. A plasticized matrix 1402 comprising at least one first excipient 1401 and at least one first solvent 1407 solvating said at least one first excipient 1401 may first be prepared. Said plasticized matrix 1402 may be disposed (e.g., placed, located, positioned, filled, etc.) in an extrusion channel 1485. Said plasticized matrix 1402 may then be extruded through an exit port 1486 of said extrusion channel 1485 by application of mechanical work to form plasticized extrudate 1403. Said plasticized extrudate 1403 may then be deposited (e.g., patterned, printed, etc.) onto an assembling stage 1490 (e.g., deposited along a path defined by the motion of said stage 1490) to form a plasticized three dimensional support structure 1404 defined by the motion of said stage 1490. Subsequently (or concurrently), said first solvent 1407 may be evaporated from said plasticized three dimensional support structure 1404 to solidify said plasticized three dimensional support structure 1404 and form a supporting solid 1400 comprising a solid, internal support structure 1405.

In some embodiments, moreover, both heating at least one first excipient and solvation of at least one first excipient may be applied to form a plasticized matrix. By way of example but not by way of limitation, both heating and solvation may be applied to minimize the amount of first solvent required for obtaining a plasticized matrix with desirable properties (e.g., desirable viscosity, etc.) for extrusion and deposition on an assembling stage.

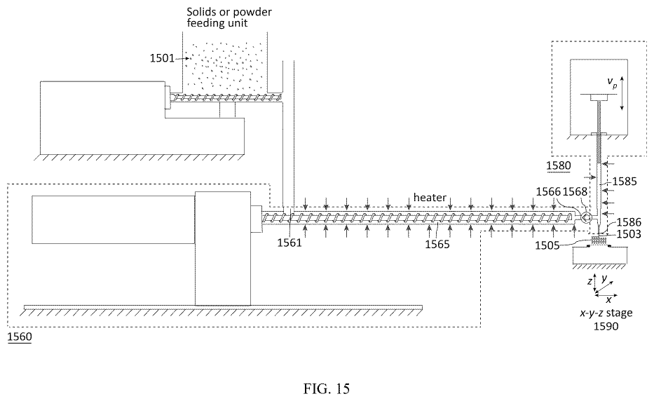

In some embodiments, moreover, forming a plasticized matrix and depositing plasticized extrudate onto an assembling stage may be integrated in a continuous process. FIG. 15 presents a non-limiting example of such a process. At least a first pharmaceutical excipient 1501 that melts upon heating may be injected (e.g., fed, delivered, etc.) into the extrusion channel 1565 of a first extruder 1560. Said first extruder's extrusion channel 1565 may terminate at at least one exit port 1566 having a valve 1568 (e.g., a check valve, a shut-off valve, etc.). In the extrusion channel 1565 the injected excipient 1501 may be heated to form a plasticized matrix. The plasticized matrix may be conveyed to the first extruder's exit port 1566 and extruded through said exit port 1566 and the valve 1568, thereby filling at least one extrusion channel 1585 of at least one second extruder 1580 with said extruded plasticized matrix. The second extruder's extrusion channel 1585 may terminate at at least one exit port 1586. The plasticized matrix in said second extruder's 1580 extrusion channel 1585 may be extruded through said exit port 1586 by application of mechanical work to form plasticized extrudate 1503. Said plasticized extrudate 1503 may then be deposited (e.g., patterned, printed, etc.) onto an assembling stage 1590 (e.g., deposited along a path defined by the motion of said stage 1590) to form a supporting solid having a three dimensional support structure 1505 defined by the motion of said stage 1590. It may be noted, moreover, that generally, a heated plasticized extrudate 1503 may also be cooled during or after deposition on an assembling stage 1590 to solidify said plasticized extrudate 1503.

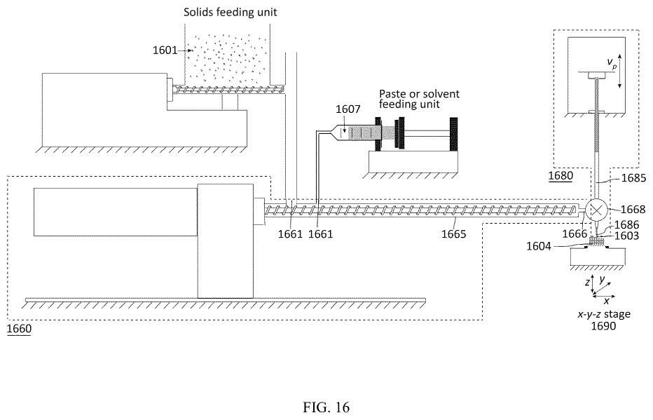

FIG. 16 presents another non-limiting example of a process where forming a plasticized matrix and depositing plasticized extrudate onto an assembling stage is integrated in a continuous process. At least a first pharmaceutical excipient 1601 and at least a first solvent 1607 may be injected into an extrusion channel 1665 of at least one first extruder 1660. Said first extruder's extrusion channel 1665 may terminate at at least one exit port 1666 having a valve 1668 (e.g., a check valve, a shut-off valve, etc.). In the first extruder's extrusion channel 1665 the injected first excipient 1601 and first solvent 1607 may be mixed to form a plasticized matrix. The plasticized matrix may be conveyed to the first extruder's exit port 1666 and extruded through said exit port 1666 and the valve 1668, thereby filling at least one extrusion channel 1685 of at least one second extruder 1680 with said extruded plasticized matrix. The second extruder's extrusion channel 1685 may terminate at at least one exit port 1686. The plasticized matrix in said second extruder's 1680 extrusion channel 1685 may be extruded through said exit port 1686 by application of mechanical work to form plasticized extrudate 1603. Said plasticized extrudate 1603 may then be deposited (e.g., patterned, printed, etc.) onto an assembling stage 1690 (e.g., deposited along a path defined by the motion of said stage 1690) to form a three dimensional support structure 1604 defined by the motion of said stage 1690. Subsequently (or concurrently), said first solvent 1607 may be evaporated from said three dimensional support structure 1604 to solidify said three dimensional support structure 1604 and form a supporting solid.

It may be noted that in some embodiments, a plasticized matrix may also be prepared outside the extrusion channel of a first extruder and then be injected (e.g., fed, delivered, disposed, etc.) into an extrusion channel of at least one first extruder. More generally, therefore, as shown in the non-limiting FIGS. 15 and 16, a non-limiting continuous process of manufacturing supporting solids herein may comprise the steps of (a) forming a plasticized matrix, said plasticized matrix disposed in an extrusion channel 1565,1665 of at least one first extruder 1560,1660. Said first extruder's extrusion channel 1565,1665 may terminate at at least one exit port 1566,1666 having a valve 1568,1668 (e.g., a check valve, a shut-off valve, etc.), (b) conveying the plasticized matrix to the first extruder's exit port 1566,1666 and extruding said plasticized matrix through said exit port 1566,1666 and the valve 1568,1668, thereby filling at least one extrusion channel 1585,1685 of at least one second extruder 1580,1680 with said extruded plasticized matrix. The second extruder's extrusion channel 1585,1685 may terminate at at least one exit port 1586,1686, (d) extruding the plasticized matrix in said second extruder's 1580,1680 extrusion channel 1585,1685 through said exit port 1586,1686 by application of mechanical work to form plasticized extrudate 1503,1603. Said plasticized extrudate 1503,1603 may then be deposited (e.g., patterned, printed, etc.) onto an assembling stage 1590,1690 (e.g., deposited along a path defined by the motion of said stage 1590,1690) to form a three dimensional support structure 1504,1604 defined by the motion of said stage 1590,1690. Subsequently (or concurrently), said three dimensional support structure 1504,1604 may be solidified to form a supporting solid.

Generally, a valve 1568,1668 at an exit port 1566,1666 of a first extruder's extrusion channel 1565,1665 may permit or allow flow of plasticized matrix from the extrusion channel 1565,1665 of said first extruder 1560,1660 into an extrusion channel 1585,1685 of a second extruder 1580,1680 while said second extruder's channel 1585,1685 is filled with plasticized matrix. Moreover, generally, said valve 1568,1668 may block (e.g., restrict, prevent, etc.) flow of plasticized matrix from an extrusion channel 1585,1685 of a second extruder 580,680 into a first extruder's extrusion channel 1565,1665 while plasticized extrudate is extruded through an exit port 1586,1686 of said second extruder's extrusion channel 1585,1685. Non-limiting examples of valves that may satisfy the above requirements comprise check valves or shut-off valves.

It may be noted, moreover, that a second extruder herein may be capable of micro-patterning (e.g., depositing, printing patterning, 3D-printing, 3D-patterning, etc.) plasticized extrudate on an assembling stage (e.g., a movable stage, etc.). The second extruder and assembling stage may therefore also be referred to herein as “micropatterning unit”, “printing unit”, “3D-micro-pattering unit”, “3D-printing unit”, and so on.

In some embodiments of the invention herein, mechanical work may be applied on a plasticized matrix by one or more rotating screws. Similarly, in some embodiments, mechanical work may be applied on a plasticized matrix by an advancing piston. It may be obvious to a person of ordinary skill in the art that more ways may exist to apply mechanical work on a plasticized matrix for extrusion through an exit port and formation of plasticized extrudate.

It may be noted, moreover, that in preferred embodiments, plasticized extrudate may be deposited (e.g., patterned, printed, etc.) on an assembling stage to form a three dimensional support structure defined by the motion of said stage at the speed of the exiting, plasticized extrudate. By way of example but not by way of limitation, in such embodiments the speed or velocity of said stage may be about the same as the speed or velocity of plasticized extrudate.

In such (and other) preferred embodiments, plasticized matrix may be extruded through an exit port of an extrusion channel at a controlled speed. By way of example but not by way of limitation, plasticized matrix may be extruded through an exit port of an extrusion channel at a controlled speed using an advancing piston. In such embodiments, the rate or speed at which plasticized matrix may be extruded through said exit port may be determined or controlled by the rate or speed at which said piston advances. In some embodiments, said piston may be driven by an electric motor enabling precise control of the rate or speed at which it advances.

It may be noted, moreover, that in some embodiments, plasticized extrudate may be substantially orderly or substantially repeatably arranged in a deposited, three dimensional support structure. Generally, plasticized extrudate may be understood as “orderly arranged” or “repeatably arranged” if such structural features as spacing between elements or segments of said plasticized extrudate, orientation of said plasticized extrudate or elements or segments thereof, etc. is/are controlled. A structural feature may be referred to as “controlled” if a standard deviation of said feature across a deposited, three dimensional support structure (or across multiple deposited, three dimensional support structures of multiple dosage forms, etc.) is smaller (or much smaller) than that in a random structure with randomly arranged plasticized extrudate. Generally, moreover, the properties of ordered structures may be controllable or better controllable than those or random structures. They may therefore be preferred.

In some embodiments, moreover, extruded plasticized matrix (e.g., plasticized extrudate, etc.) may comprise extruded plasticized fiber. In such embodiments, extruded plasticized fiber may be deposited onto a fiber assembling stage to form a three dimensional fiber structural framework.

By way of example but not by way of limitation, extruded plasticized fiber may be deposited onto a fiber assembling stage to form a three dimensional structural framework of criss-crossed stacked layers of one or more fibers. In the invention herein, a three dimensional structural framework of criss-crossed stacked layers of one or more fibers may generally be understood as “three-dimensional structural framework of one or more fibers forming fiber layers stacked in a cross-ply arrangement”.

It may be obvious to a person of ordinary skill in the art that in such embodiments, a three dimensional support structure may comprise a three dimensional structural network or framework of criss-crossed stacked layers of one or more fibers. Generally, moreover, such fibrous structures may have free spacings defining free spaces between fibers or fiber segments.

In some embodiments, moreover, a supporting solid herein may comprise an exterior or outer surface, an internal, three-dimensional structural framework of criss-crossed stacked layers of one or more fibers contiguous with and terminating at said outer surface, and one or more internal, open free spaces. In the invention herein, a three dimensional structural framework of criss-crossed stacked layers of one or more fibers may generally be understood to as “three-dimensional structural framework of one or more fibers forming fiber layers stacked in a cross-ply arrangement”, and so on.

Thus, in some embodiments, as shown in the non-limiting FIGS. 12-14, a supporting solid 1200,1300,1400 herein may comprise an exterior or outer surface 1206,1306,1406, an internal, three-dimensional solid support structure 1205,1305,1405 contiguous with and terminating at said outer surface 1206,1306,1406, and one or more internal free spaces 1230,1330,1430. Thus, generally, a supporting solid may be considered as “envelope” or “outer volume” of an internal support structure 1205,1305,1405.

In preferred embodiments, one or more free spaces in a supporting solid may be filled with a gas, such as ambient air.

In preferred embodiments, moreover, one or more free spaces may be substantially open to an exterior surface of a supporting solid. Thus, in some embodiments, one or more free spaces may have at least one open end contiguous with and terminating at an exterior or outer surface of a supporting solid. In some embodiments, moreover, one or more free spaces may have at least two open ends contiguous with and terminating at an exterior or outer surface of a supporting solid. If one or more free spaces are open to an exterior surface of a supporting solid, physiological fluid may percolate into said channels upon contact with said physiological fluid.

In some embodiments, moreover, one or more free spaces may be substantially connected through an exterior dimension of a supporting solid.

In some embodiments, first solvent may be evaporated or boiled away from a deposited three dimensional structural framework by reducing the pressure (e.g., by reducing the pressure of the gas) surrounding the plasticized extrudate to a pressure about equal to or slightly greater or slightly lower (e.g., smaller) than the vapor pressure of the first solvent (e.g., the vapor pressure of the first solvent at the temperature of the solvent in the deposited framework).

In some embodiments, the pressure of the gas surrounding a deposited three dimensional structural framework during first solvent removal may be less than the atmospheric pressure (e.g., less than 1 bar). This includes, but is not limited to a pressure of the gas surrounding a deposited three dimensional structural framework during first solvent removal no greater than 800 mbar, or no greater than 700 mbar, or no greater than 600 mbar, or no greater than 500 mbar, or no greater than 400 mbar, or no greater than 300 mbar, or no greater than 200 mbar, or no greater than 100 mbar, or no greater than 75 mbar.

In some embodiments, moreover, a reduced pressure (or vacuum) may be maintained for prolonged time, such as longer than 1 minute, or longer than 2 minutes, or longer than 5 minutes, or longer than 10 minutes, or longer than 20 minutes, or longer than 50 minutes, or longer than 100 minutes, or longer than 200 minutes, or longer than 500 minutes, or longer than 1000 minutes. Such “vacuum drying” or “removing solvent using a vacuum” or “removing solvent by reducing pressure”, etc. may allow more uniform shrinkage of a deposited framework during first solvent removal.

It may be obvious to a person of ordinary skill in the art that more ways of evaporating first solvent from a deposited three dimensional structural framework exist. Such ways include, but are not limited to blowing a gas, such as air, on or through the framework, and so on. All these methods of first solvent evaporation that are obvious to a person of ordinary skill in the art are included in this invention.

Moreover, it may be obvious to a person of ordinary skill in the art that more ways of manufacturing supporting solids herein exist. All such methods of manufacturing supporting solids obvious to a person of ordinary skill in the art are included in this invention.

### (b) Embodiments Related to Coating a Three Dimensional Support Structure of a Supporting Solid

In some embodiments of the invention herein, an internal, three dimensional support structure of a supporting solid as disclosed herein (e.g., a supporting three dimensional structural framework of fibers, etc.) may be coated with a coating.

In some embodiments, coating an internal, three-dimensional support structure of a supporting solid may comprise the step of dipping said supporting solid into a coating solution. By way of example but not by way of limitation, a coating solution may comprise at least a third pharmaceutical excipient (e.g., at least a pharmaceutical excipient) and at least a third solvent (e.g., at least a solvent) solvating said third excipient. In other words a coating solution may comprise a third excipient dissolved in a third solvent.

In some embodiments, furthermore, coating an internal support structure of a supporting solid may also comprise the step of withdrawing said supporting solid from a coating solution. This includes, but is not limited to withdrawing a supporting solid from a coating solution to form a coated supporting solid having a three-dimensional support structure substantially coated (e.g., substantially encapsulated, substantially surrounded, substantially covered, substantially enclosed, etc.) by a coating.

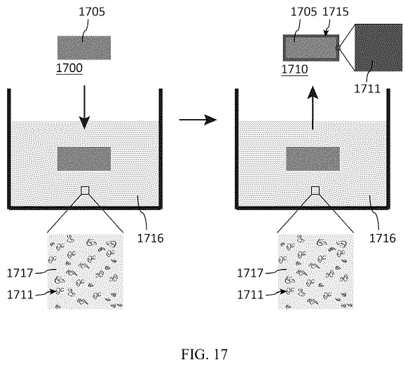

FIG. 17 presents a non-limiting example of a method by which a three-dimensional support structure 1705 of a supporting solid 1700 (e.g., a supporting three dimensional structural framework of fibers, etc.) may be coated (e.g., substantially encapsulated, substantially surrounded, substantially covered, substantially enclosed, etc.) by a coating 1715. Said supporting solid 1700 may first be dipped into (e.g., exposed to, immersed into, etc.) a coating solution 1716. Said coating solution 1716 may comprise at least a third pharmaceutical excipient 1711 and at least a third solvent 1717 solvating said third excipient 1711. Subsequently (or concurrently), said supporting solid 1700 may be withdrawn from said coating solution 1716 to form a coated supporting solid 1710 having a three-dimensional support structure 1705 substantially coated (e.g., substantially encapsulated, substantially surrounded, substantially covered, substantially enclosed, etc.) by a coating 1715.

In some embodiments, moreover, a wet supporting solid may be formed upon withdrawing a supporting solid from a coating solution.

In some embodiments, a wet supporting solid may comprise an internal, three dimensional support structure and a coating solution attached to (e.g., substantially surrounding, substantially covering, adhered to, added to, etc.) said internal, three dimensional support structure. By way of example but not by way of limitation, a wet supporting solid may comprise an internal, three dimensional support structure substantially surrounded by a coating solution. Moreover, a wet supporting solid may comprise an internal, three dimensional support structure substantially surrounded by a layer of a coating solution.

In some embodiments, moreover, coating an internal support structure may further comprise evaporating third solvent from a wet supporting solid. More specifically, in some embodiments coating an internal support structure may further comprise evaporating third solvent from a wet supporting solid to form a coated supporting solid (e.g., to solidify a coating solution surrounding an internal support structure and form a coated supporting solid) having an internal, three dimensional support structure substantially coated by a solid coating.

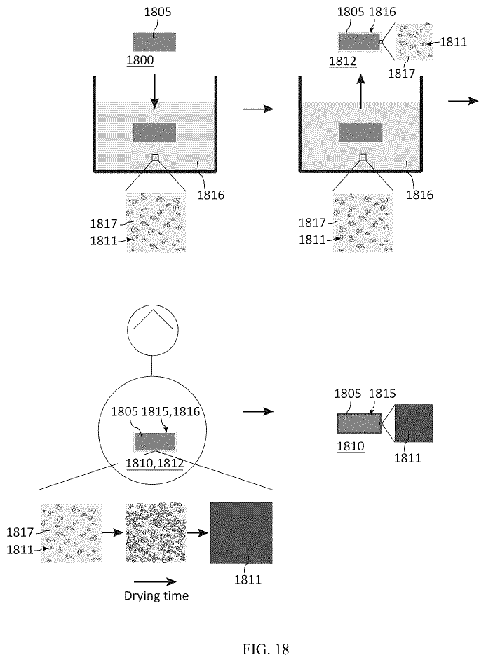

FIG. 18 presents another non-limiting example of a method by which a three-dimensional support structure 1805 of a supporting solid 1800 may be coated (e.g., substantially encapsulated, substantially surrounded, substantially covered, substantially enclosed, etc.) by a coating 1815. Said supporting solid 1800 may first be dipped into (e.g., exposed to, immersed into, etc.) a coating solution 1816. Said coating solution 1816 may comprise at least a third pharmaceutical excipient 1811 and at least a third solvent 1817 solvating said third pharmaceutical excipient 1811. Subsequently (or concurrently), said supporting solid 1800 may be withdrawn from said coating solution 1816 to form a wet supporting solid 1812 having a three-dimensional support structure 1805 substantially surrounded by (e.g., substantially surrounded by a layer of) said coating solution 1816. Subsequently (or concurrently), third solvent 1817 may be evaporated from said wet supporting solid 1812 (e.g., from said coating solution 1816 substantially surrounding said internal support structure 1805, from said layer of coating solution 1816 substantially surrounding said internal support structure 1805, etc.) to form a coated supporting solid 1810 having an internal, three-dimensional support structure 1805 substantially coated by a solid coating 1815.

Furthermore, in some embodiments, a supporting solid herein may comprise an exterior or outer surface, an internal, three-dimensional solid support structure contiguous with and terminating at said outer surface, and one or more internal, open support-structure-free spaces.

In some embodiments, accordingly, upon dipping a supporting solid into a coating solution, said coating solution may percolate one or more internal, open support-structure-free spaces of said supporting solid.

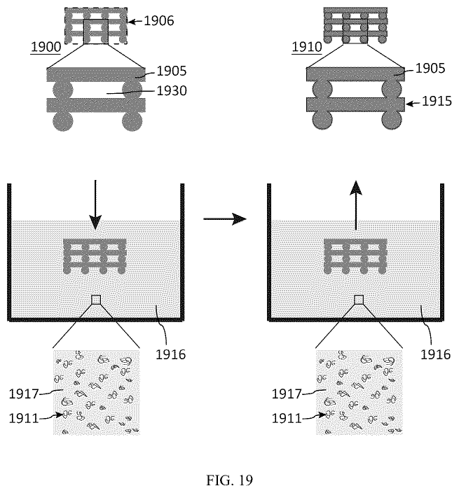

FIG. 19 presents a non-limiting example of a method by which a three-dimensional solid support structure of a supporting solid 1900 comprising an exterior or outer surface 1906, an internal, three-dimensional solid support structure 1905 contiguous with and terminating at said outer surface 1906, and one or more internal, open support-structure-free spaces 1930 may be substantially coated (e.g., substantially encapsulated, substantially surrounded, substantially coated, substantially enclosed, etc.) by a coating 1915. Said supporting solid 1900 may first be dipped into (e.g., exposed to, immersed into, etc.) a coating solution 1916 comprising at least a third pharmaceutical excipient 1911 and at least a third solvent 1917 solvating said third pharmaceutical excipient 1911. Said coating solution 1916 may then percolate (e.g., fill, etc.) one or more open support-structure-free spaces 1930 of said supporting solid 1900. Subsequently (or concurrently), said supporting solid 1900,1910 may be withdrawn from said coating solution 1916 to form a coated supporting solid 1910 having a three-dimensional support structure 1905 substantially coated by a coating 1915.

In some embodiments, an internal, three dimensional support structure may comprise a three-dimensional structural framework of one or more fibers.

In some embodiments, an internal, three dimensional support structure may comprise criss-crossed stacked layers of one or more fibers.

In some embodiments, moreover, upon dipping a supporting solid into a coating solution, said coating solution may percolate one or more fiber-free spaces between adjoining fiber segments of criss-crossed stacked layers of one or more fibers.

FIG. 19 also presents another non-limiting example of a method by which a three-dimensional solid support structure 1905 of a supporting solid 1900 comprising an exterior or outer surface 1906, an internal, three-dimensional structural framework of criss-crossed stacked layers of one or more fibers 1905 contiguous with and terminating at said outer surface 1906, and one or more internal, open fiber-free spaces 1930 may be substantially coated (e.g., substantially encapsulated, substantially surrounded, substantially coated, substantially enclosed, etc.) by a coating 1915. Said supporting solid 1900 (e.g., the supporting, three dimensional structural framework of crissed-crossed stacked layers of one or more fibers 1905) may first be dipped into (e.g., exposed to, immersed into, etc.) a coating solution 1916 comprising at least a third pharmaceutical excipient 1911 and at least a third solvent 1917 solvating said third pharmaceutical excipient 1911. Said coating solution 1916 may then percolate (e.g., fill, etc.) one or more open fiber-free spaces 1930 of said supporting solid 1900 (e.g., one or more free spaces 1930 between fibers or fiber segments of the supporting, three dimensional fiber structural framework 1905). Subsequently (or concurrently), said supporting solid 1900 may be withdrawn from said coating solution 1916 to form a coated supporting solid 1910 having a three-dimensional structural framework of criss-crossed stacked layers of fibers 1905 substantially coated by a coating 1915.

In some embodiments, a supporting solid comprising an exterior or outer surface, an internal, three-dimensional solid support structure contiguous with and terminating at said outer surface, and one or more internal, open support-structure-free spaces may be withdrawn from a coating solution to form a wet supporting solid having an internal, three-dimensional support structure and one or more internal, open support-structure-free spaces filled with said coating solution. In such embodiments, moreover, coating an internal support structure may further comprise evaporating solvent (e.g., third solvent) from a wet supporting solid to form a coated supporting solid (e.g., to solidify said coating solution within one or more support-structure-free spaces and form a coated supporting solid) having an internal, three dimensional support structure substantially coated by a solid coating.

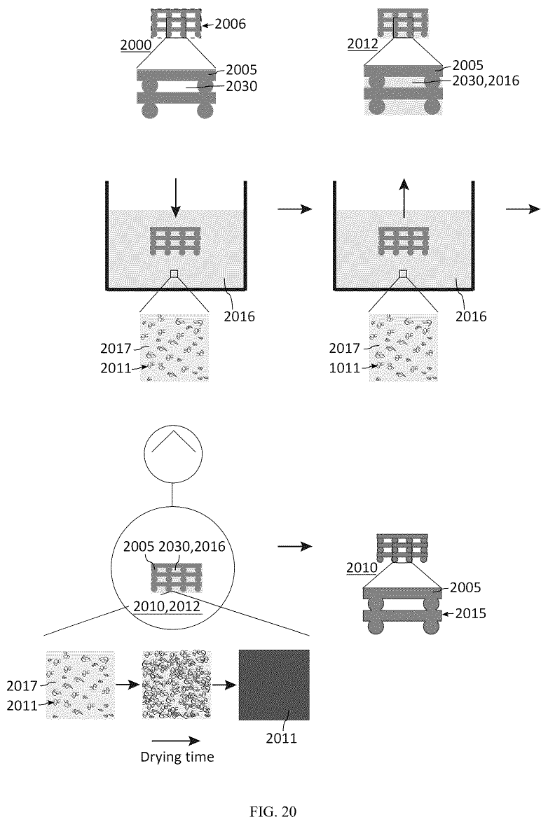

FIG. 20 presents another non-limiting example of a method by which a three-dimensional solid support structure 2005 of a supporting solid 2000 comprising an exterior or outer surface 2006, an internal, three-dimensional solid support structure 2005 contiguous with and terminating at said outer surface 2006, and one or more internal, open support-structure-free spaces 2030 may be substantially coated (e.g., substantially encapsulated, substantially surrounded, substantially covered, substantially enclosed, etc.) by a coating 2015. Said supporting solid 2000 may first be dipped into (e.g., exposed to, immersed into, etc.) a coating solution 2016 comprising at least a third pharmaceutical excipient 2011 and at least a third solvent 2017 solvating said third pharmaceutical excipient 2011. Said coating solution 2016 may then percolate (e.g., fill, etc.) one or more open support-structure-free spaces 2030 of said supporting solid 2000. Subsequently (or concurrently), said supporting solid 2000 may be withdrawn from said coating solution 2016 to form a wet supporting solid 2012 having an internal, three-dimensional support structure 2005 and one or more internal, open support-structure-free spaces 2030 substantially or partially filled with said coating solution 2016. Subsequently (or concurrently), third solvent 2017 may be evaporated from said wet supporting solid 2012 (e.g., from said coating solution 2016 within one or more support-structure-free spaces 2030, etc.) to form a coated supporting solid 2010 (e.g., to solidify said coating solution 2016 within one or more support-structure-free spaces 2030 and form a coated supporting solid 2010) having a three-dimensional support structure 2005 substantially coated by a solid coating 2015.

In some embodiments, a wet supporting solid may have an internal, three-dimensional structural framework one or more fibers (e.g., criss-crossed stacked layers of one or more fibers) and one or more internal, open fiber-free spaces filled with a coating solution.

Thus, in some embodiments a supporting solid comprising an exterior or outer surface, an internal, three-dimensional structural framework of one or more fibers (e.g., criss-crossed stacked layers of one or more fibers) contiguous with and terminating at said outer surface, and one or more internal, open fiber-free spaces may be withdrawn from a coating solution to form a wet supporting solid having an internal, three-dimensional structural framework one or more fibers (e.g., criss-crossed stacked layers of one or more fibers) and one or more internal, open fiber-free spaces filled with said coating solution.

In such embodiments, moreover, third solvent may be evaporated from a wet supporting solid to form a coated supporting solid (e.g., to solidify a coating solution within one or more fiber-free spaces and form a coated supporting solid) having internal, criss-crossed stacked layers of one or more fibers substantially coated by a solid coating. In some embodiments, therefore, coating an internal support structure comprising criss-crossed stacked layers of one or more fibers may further comprise evaporating solvent from a wet supporting solid to form a coated supporting solid having internal, criss-crossed stacked layers of one or more fibers substantially encapsulated by a solid coating.

FIG. 20 also presents a non-limiting example of a method by which a three-dimensional solid support structure 2005 of a supporting solid 2000 comprising an exterior or outer surface 2006, an internal, three-dimensional structural framework of criss-crossed stacked layers of one or more fibers 2005 contiguous with and terminating at said outer surface 2006, and one or more internal, open fiber-free spaces 2030 may be substantially coated (e.g., substantially encapsulated, substantially surrounded, substantially covered, substantially enclosed, etc.) by a coating 2015. Said supporting solid 2000 may first be dipped into (e.g., exposed to, immersed into, etc.) a coating solution 2016 comprising at least a third pharmaceutical excipient 2011 and at least a third solvent 2017 solvating said third excipient 2017. Said coating solution 2016 may then percolate (e.g., fill, etc.) one or more open fiber-free spaces 2030 of said supporting solid 2000. Subsequently (or concurrently), said supporting solid 2000 may be withdrawn from said coating solution 2016 to form a wet supporting solid 2012 having an internal, three-dimensional structural framework of criss-crossed stacked layers of one or more fibers 2005 and one or more internal, open fiber-free spaces 2030 substantially filled with said coating solution 2016. Subsequently (or concurrently), third solvent 2017 may be evaporated from said wet supporting solid 2012 (e.g., from said coating solution 2016 within one or more fiber-free spaces 2030, etc.) to form a coated supporting solid 2010 (e.g., to solidify said coating solution 2016 within one or more fiber-free spaces 2030 and form a coated supporting solid 2010) having a three-dimensional structural framework of criss-crossed stacked layers of one or more fibers 2005 substantially coated by a solid coating 2015.

In some embodiments, moreover, after solvent evaporation solid (or semi-solid, etc.) coating may close (e.g., bridge, etc.) one or more support-structure-free spaces between segments of a coated, internal three-dimensional support structure of a supporting solid.

In some embodiments, therefore, a needle or pin may be pushed into or through one or more support-structure-free spaces of a coated supporting solid to open (e.g., form, etc.) one or more internal, coated-support-structure-free spaces within said coated supporting solid. This includes, but is not limited to pushing a needle or pin into or through one or more support-structure-free spaces between segments of a coated, internal three dimensional support structure to open (e.g., form, etc.) one or more internal, coated-support-structure-free spaces within a coated supporting solid. This also includes, but is not limited to pushing a needle or pin into or through one or more support-structure-free spaces between segments of a coated, internal three dimensional support structure to form a coated supporting solid having an outer surface, a coated, internal three-dimensional support structure contiguous with and terminating at said outer surface, and one or more internal, open coated-support-structure-free spaces.

Similarly, after solvent evaporation solid (or semi-solid, etc.) coating may close (e.g., bridge, etc.) one or more fiber-free spaces between fiber segments of a three-dimensional structural framework of criss-crossed stacked layers of one or more coated fibers.

In some embodiments, therefore, a needle or pin may be pushed into or through one or more fiber-free spaces between adjoining fiber segments of criss-crossed stacked layers of one or more coated fibers to open (e.g., form) one or more internal, coated-fiber-free spaces in a coated supporting solid. In some embodiments, moreover, said needle may advance transversally (e.g., orthogonally) to the layers of said criss-crossed stacked layers of one or more coated fibers.

In some embodiments, solvent (e.g., third solvent) may be evaporated or boiled away from a coating solution by reducing the pressure (e.g., by reducing the pressure of the gas) surrounding the coating solution to a pressure about equal to or slightly greater or lower (e.g., smaller) than the vapor pressure of the solvent (e.g., the vapor pressure of the solvent at the temperature of the solvent in the coating solution).

In some embodiments, moreover, the pressure of the gas surrounding a coating solution during solvent removal may be less than the atmospheric pressure (e.g., less than 1 bar). This includes, but is not limited to a pressure of the gas surrounding a coating solution during solvent removal no greater than 800 mbar, or no greater than 700 mbar, or no greater than 600 mbar, or no greater than 500 mbar, or no greater than 400 mbar, or no greater than 300 mbar, or no greater than 200 mbar, or no greater than 100 mbar, or no greater than 75 mbar.

In some embodiments, furthermore, a reduced pressure (or vacuum) may be maintained for prolonged time, such as longer than 1 minute, or longer than 2 minutes, or longer than 5 minutes, or longer than 10 minutes, or longer than 20 minutes, or longer than 50 minutes, or longer than 100 minutes, or longer than 200 minutes, or longer than 500 minutes, or longer than 1000 minutes. Such “vacuum drying” or “removing solvent using a vacuum” or “removing solvent by reducing pressure”, etc. may, for example, allow more uniform shrinkage of a deposited framework during solvent removal.

It may be obvious to a person of ordinary skill in the art that more ways of evaporating solvent from a deposited three dimensional structural framework exist. Such ways include, but are not limited to blowing a gas, such as air, on or through the framework, and so on. All these methods of solvent evaporation that are obvious to a person of ordinary skill in the art are included in this invention.

Also, it may be obvious to a person of ordinary skill in the art that many more methods of coating a supporting solid could be presented. All of methods obvious to a person of ordinary skill in the art are within the spirit and scope of this invention.

### (c) Embodiments Related to Adding Drug-Containing Matter to Supporting Solid

After preparation of a supporting solid or a coated supporting solid, drug-containing matter may be added or attached to a support structure or a coated support structure of said supporting solid or said coated supporting solid. In the invention herein, the mention of “adding or attaching drug-containing matter to a supporting solid” also includes “adding or attaching drug-containing matter to a coated supporting solid”. Thus, in embodiments related to adding or attaching drug-containing matter (e.g., drug-containing plasticized matrix, drug-containing solid, a drug-containing solution, a drug-containing dispersion, and so on) the terms “supporting solid” and “coated supporting solid”, “support structure” and “coated support structure”, “framework” and “coated framework”, “three dimensional structural framework of one or more fibers” and “coated three dimensional structural framework of one or more fibers”, “three dimensional structural framework of one or more elements” and “coated three dimensional structural framework of one or more elements”, “three dimensional fiber structural framework” and “coated three dimensional fiber structural framework”, “supporting fibrous structural framework” and “coated supporting fibrous structural framework”, and so on are used interchangeably.

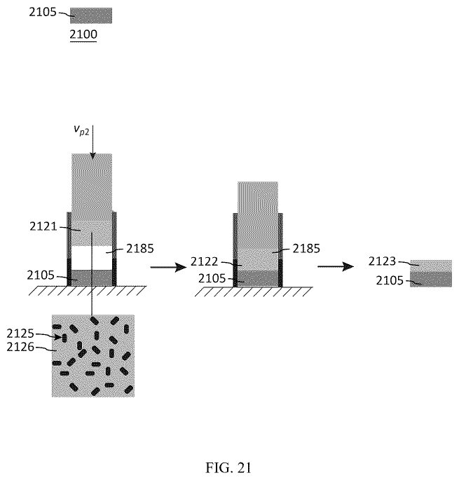

FIG. 21 presents a non-limiting method by which drug-containing matter may be added to a support structure 2105 of a supporting solid 2100. First, drug-containing plasticized matrix 2121 comprising at least one drug 2125 and at least one second excipient 2126 may be prepared. Said drug-containing plasticized matrix 2121 may (subsequently or concurrently) be disposed (e.g., placed, located, positioned, filled, etc.) in an extrusion channel 2185. Said drug-containing plasticized matrix 2121 may then be extruded through said extrusion channel 2185 by application of mechanical work. A controlled amount of said extruded drug-containing plasticized matrix 2122 may then be added to (e.g., attached to, adhered to, combined with, binded to, etc.) said support structure 2105 (e.g., a surface or a deposition surface of said support structure 2105, etc.). Subsequently (or concurrently), the added drug-containing plasticized matrix 2122 (e.g., the extruded drug-containing plasticized matrix 2122 added to said support structure 2105, etc.) may be solidified to form drug-containing solid 2123 attached to said support structure 2105.

In some embodiments, a drug-containing plasticized matrix may be prepared by heating a composition comprising at least one drug and at least one second excipient. It may be noted, moreover, that generally, a heated drug-containing plasticized matrix may be cooled during or after adding to a support structure to solidify said drug-containing plasticized matrix.

In some embodiments, however, a desirable pharmaceutical composition may need to be processed at lower temperatures. In such (and other) embodiments, drug-containing plasticized matrix may be prepared by solvating at least one second excipient with a second solvent. In such embodiments, a plasticized matrix may comprise a composition or mixture comprising at least one drug, at least one second excipient, and at least a second solvent solvating said second excipient. In such (and similar) embodiments, moreover, second solvent may be evaporated from drug-containing plasticized matrix during or after adding to a support structure to solidify said drug-containing plasticized matrix.

In some embodiments, moreover, it may be desirable to minimize the amount of second solvent used for obtaining a drug-containing plasticized matrix with desirable properties (e.g., desirable viscosity, etc.) for extrusion and addition or deposition on a support structure. In such (and similar or other) embodiments, both heating a composition or mixture comprising at least one drug and at least one second excipient and solvation of said at least one second excipient with a second solvent may be applied to form a drug-containing plasticized matrix.

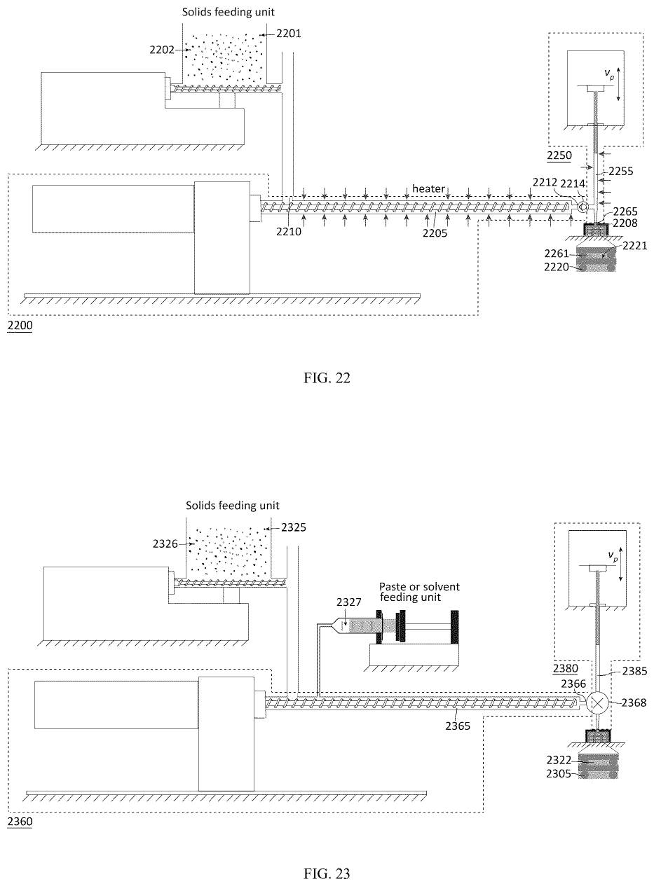

In some embodiments, moreover, forming a drug-containing plasticized matrix and adding drug-containing plasticized matrix to a support structure of a supporting solid may be integrated in a continuous process. FIG. 22 presents a non-limiting example of such a process. At least a drug 2225 and at least a second pharmaceutical excipient 2226 that melts upon heating may be injected (e.g., fed, delivered, etc.) into the extrusion channel 2265 of a third extruder 2260. Said third extruder's extrusion channel 2265 may terminate at at least one exit port 2266 having a valve 2268 (e.g., a check valve, shut-off valve, etc.). In the extrusion channel 2265 the injected drug 2225 and second excipient 2226 may be heated to form a drug-containing plasticized matrix. The drug-containing plasticized matrix may be conveyed to the third extruder's exit port 2266 and extruded through said exit port 2266 and the valve 2268, thereby filling at least one extrusion channel 2285 of at least one fourth extruder 2280 with said extruded drug-containing plasticized matrix. A controlled amount of said drug-containing plasticized matrix may then be extruded through said extrusion channel 2285 and added to (e.g., attached to, adhered to, combined with, binded to, bonded to, etc.) a support structure 2205 (e.g., a surface or a deposition surface of a support structure 2205) by application of mechanical work. Subsequently (or concurrently), the added drug-containing plasticized matrix 2261 (e.g., the drug-containing plasticized matrix 2261 added to said support structure 2205) may be solidified to form drug-containing solid attached to said support structure 2205.

FIG. 23 presents another non-limiting example of a continuous process of adding drug-containing matter to a support structure herein. At least a drug 2325, at least a second pharmaceutical excipient 2326 and at least a second solvent 2327 may injected into the extrusion channel 2365 of at least one third extruder 2360. Said third extruder's extrusion channel 2365 may terminate at at least one exit port 2366 having a valve 2368 (e.g., a check valve, shut-off valve, etc.). In the third extruder's extrusion channel 2365 the injected drug 2325, second excipient 2326 and second solvent 2327 may be mixed to form a drug-containing plasticized matrix. The drug-containing plasticized matrix may be conveyed to the third extruder's exit port 2366 and extruded through said exit port 2366 and the valve 2368, thereby filling at least one extrusion channel 2385 of at least one fourth extruder 2380 with said extruded drug-containing plasticized matrix. A controlled amount of said drug-containing plasticized matrix may then be extruded through said extrusion channel 2385 and added to (e.g., attached to, adhered to, combined with, binded to, bonded to, etc.) a support structure 2305 (e.g., a surface or a deposition surface of a support structure 2305) by application of mechanical work. Subsequently (or concurrently), said second solvent 2327 may be evaporated from said added drug-containing plasticized matrix 2322 (e.g., the drug-containing plasticized matrix 2322 added to said support structure 2305) to solidify said added drug-containing plasticized matrix 2322 and form a drug-containing solid attached to (e.g., combined with, joined with, connected to, bonded to, immovably attached to, etc.) said support structure 2305.

It may be noted that in some embodiments, a drug-containing plasticized matrix may also be prepared outside the extrusion channel of a third extruder and then be injected (e.g., fed, delivered, disposed, etc.) into an extrusion channel of at least one third extruder. More generally, therefore, as shown in the non-limiting FIGS. 22 and 23, a non-limiting continuous process of adding drug-containing matter to a support structure herein may comprise the steps of (a) forming a drug-containing plasticized matrix, said plasticized matrix disposed in an extrusion channel 2265,2365 of at least one third extruder 2260,2360. Said third extruder's extrusion channel 2265,2365 may terminate at at least one exit port 2266,2366 having a valve 2268,2368 (e.g., a check valve, a shut-off valve, etc.), (b) conveying the drug-containing plasticized matrix to the third extruder's exit port 2266,2366 and extruding said drug-containing plasticized matrix through said exit port 2266,2366 and the valve 2268,2368, thereby filling at least one extrusion channel 2285,2385 of at least one fourth extruder 2280,2380 with said extruded drug-containing plasticized matrix, (c) extruding a controlled amount of said drug-containing plasticized matrix through said extrusion channel 2285,2385 and adding to (e.g., attaching to, adhering to, combining with, binding to, bonding to, etc.) a support structure 2205,2305 (e.g., a surface or a deposition surface of a support structure 2205,2305) by application of mechanical work, and (d) solidifying said added drug-containing plasticized matrix 2222,2322 and form a drug-containing solid attached to (e.g., combined with, joined with, connected to, bonded to, immovably attached to, etc.) said support structure 2205,2305.

Generally, a valve 2268,2368 at an exit port 2266,2366 of a third extruder's extrusion channel 2265,2365 may permit or allow flow of drug-containing plasticized matrix from the extrusion channel 2265,2365 of third extruder 2260,2360 into an extrusion channel 2285,2385 of a fourth extruder 2280,2380 while said fourth extruder's channel 2285,2385 is filled with drug-containing plasticized matrix. Moreover, generally, said valve 2268,2368 may block (e.g., restrict, prevent, etc.) flow of drug-containing plasticized matrix from an extrusion channel 2285,2385 of a fourth extruder 2280,2380 into a third extruder's extrusion channel 2265,2365 while drug-containing plasticized matrix is extruded through said fourth extruder's extrusion channel 2285,2385. Non-limiting examples of valves that may satisfy the above requirements comprise a check valves and/or shut-off valves, etc.

It may be noted, moreover, that the terms “third extruder” and “fourth extruder” are used herein only to distinguish at least two extruders. The “third extruder” and the “fourth extruder” may be named differently also.

In some embodiments, moreover, a supporting solid herein may comprise an exterior or outer surface an internal, three-dimensional solid support structure contiguous with and terminating at said outer surface and one or more internal, open free spaces.

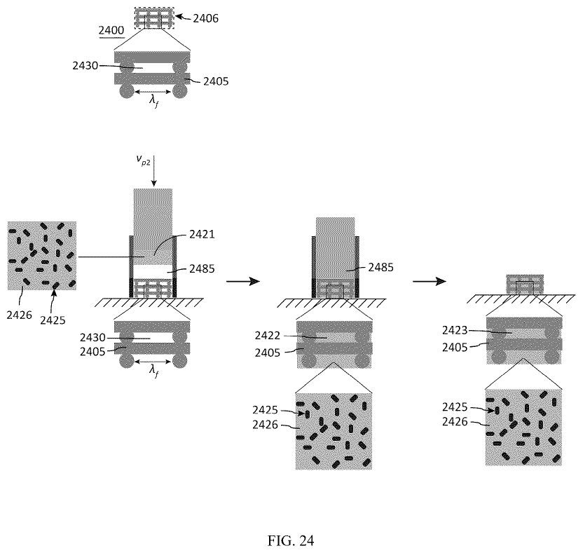

FIG. 24 presents a non-limiting method of adding drug-containing matter to an internal structure 2405 of such a supporting solid 2400 comprising an exterior or outer surface 2406, an internal, three-dimensional solid support structure 2405 contiguous with and terminating at said outer surface 2406, and internal, open free spaces 2430. First, drug-containing plasticized matrix 2421 comprising at least one drug 2425 and at least one second excipient 2426 may be prepared. Said drug-containing plasticized matrix 2421 may (subsequently or concurrently) be disposed (e.g., placed, located, positioned, filled, etc.) in an extrusion channel 2485. Said drug-containing plasticized matrix 2421 may then be extruded through said extrusion channel 2485 by application of mechanical work. A controlled amount of said extruded drug-containing plasticized matrix 2422 may be extruded (e.g., transported, squeezed, filled, etc.) into one or more internal, open free spaces 2430 of said supporting solid 2400 by application of mechanical work and added to (e.g., attached to, adhered to, combined with, binded to, bonded to, etc.) said support structure 2405 (e.g., a surface or a deposition surface of said support structure 2405). Subsequently (or concurrently), drug-containing, plasticized matrix 2422 in one or more free spaces 2430 may be solidified to form drug-containing solid 2423 attached to said support structure 2405 within one or more free spaces 2430 of said supporting solid 2400.

Furthermore, in some embodiments, an internal structure may comprise a three-dimensional structural framework of criss-crossed stacked layers of one or more fibers. Similarly, a supporting solid may comprise an exterior or outer surface, an internal, three-dimensional structural framework of criss-crossed stacked layers of one or more fibers contiguous with and terminating at said outer surface, and one or more internal, open free spaces.

FIG. 24 also presents a non-limiting method of attaching drug-containing matter to an internal structure 2405 of such a supporting solid 2400 comprising an exterior or outer surface 2406, an internal, three-dimensional structural framework of criss-crossed stacked layers of one or more fibers 2405 contiguous with and terminating at said outer surface 2406, and one or more internal, open free spaces 2430. First, drug-containing plasticized matrix 2421 comprising at least one drug 2425 and at least one second excipient 2426 may be prepared. Said drug-containing plasticized matrix 2421 may (subsequently or concurrently) be disposed (e.g., placed, located, positioned, filled, etc.) in an extrusion channel 2485. Said drug-containing plasticized matrix 2421 may then be extruded through said extrusion channel 2485 by application of mechanical work. A controlled amount of said extruded drug-containing plasticized matrix 2422 may be extruded (e.g., transported, squeezed, filled, etc.) into one or more internal, open free spaces 2430 of said supporting solid 2400 (e.g., into one or more free spaces between one or more fibers or fiber segments of said three dimensional fibrous structural framework 2405) by application of mechanical work and added to (e.g., attached to, adhered to, combined with, binded to, bonded to, etc.) said three-dimensional structural framework of one or more fibers 2405 (e.g., a surface or a deposition surface of said three dimensional structural framework of one or more fibers 2405). Subsequently (or concurrently), drug-containing plasticized matrix 2422 in one or more free spaces 2430 may be solidified to form drug-containing solid 2423 attached to said three dimensional structural framework of one or more fibers 2405 within one or more free spaces 2430 of said supporting solid 2400.

As described above, a drug-containing plasticized matrix may generally be prepared by heating and/or solvating a composition comprising at least one drug and at least one second excipient. Similarly, an extruded drug-containing plasticized matrix may generally be solidified by cooling it and/or evaporating solvent from it.

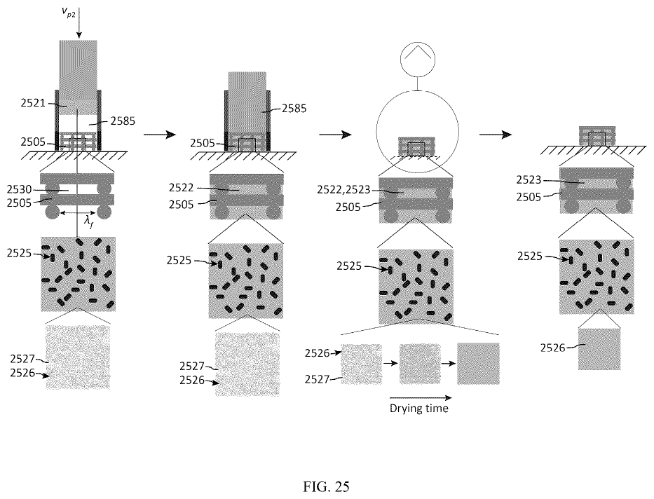

FIG. 25 presents another non-limiting method of adding drug-containing matter to an internal structure 2505 of a supporting solid 2500 comprising an exterior or outer surface 2506, an internal, three-dimensional solid support structure 2505 contiguous with and terminating at said outer surface 2506, and internal, open free spaces 2530. First, drug-containing plasticized matrix 2521 comprising at least one drug 2525, at least one second excipient 2526, and at least one second solvent 2527 that solvates at least one second excipient 2526 may be prepared. Said drug-containing plasticized matrix 2521 may (subsequently or concurrently) be disposed (e.g., placed, located, positioned, filled, etc.) in an extrusion channel 2585. Said drug-containing plasticized matrix 2521 may then be extruded through said extrusion channel 2585 by application of mechanical work. A controlled amount of said extruded drug-containing plasticized matrix 2522 may be extruded (e.g., transported, squeezed, filled, etc.) into one or more internal, open free spaces 2530 of said supporting solid 2500 by application of mechanical work and added to (e.g., attached to, adhered to, combined with, binded to, bonded to, etc.) said support structure 2505 (e.g., a surface or a deposition surface of said support structure 2505). Subsequently (or concurrently), second solvent 2527 may be evaporated from drug-containing, plasticized matrix 2522 in one or more free spaces 2530 to solidify said drug-containing plasticized matrix 2522 and form drug-containing solid 2523 attached to said support structure 2505 (e.g., drug-containing solid 2523 attached to said support structure 2505 within one or more free spaces 2530 of said supporting solid 2500).

FIG. 25 also presents a non-limiting method of attaching drug-containing matter to an internal structure 2505 of a supporting solid 2500 comprising an exterior or outer surface 2506, an internal, three-dimensional structural framework of criss-crossed stacked layers of one or more fibers 2505 contiguous with and terminating at said outer surface 2506, and one or more internal, open free spaces 2530. First, drug-containing plasticized matrix 2521 comprising at least one drug 2525, at least one second excipient 2526, and at least one second solvent 2527 that solvates at least one second excipient 2526 may be prepared. Said drug-containing plasticized matrix 2521 may (subsequently or concurrently) be disposed (e.g., placed, located, positioned, filled, etc.) in an extrusion channel 2585. Said drug-containing plasticized matrix 2521 may then be extruded through said extrusion channel 2585 by application of mechanical work. A controlled amount of said extruded drug-containing plasticized matrix 2522 may be extruded (e.g., transported, squeezed, filled, etc.) into one or more internal, open free spaces 2530 of said supporting solid 2500 by application of mechanical work and added to (e.g., attached to, adhered to, combined with, binded to, bonded to, etc.) said three-dimensional structural framework of one or more fibers 2505 (e.g., a surface or a deposition surface of said three dimensional structural framework of one or more fibers 2505). Subsequently (or concurrently), second solvent 2527 may be evaporated from drug-containing, plasticized matrix 2522 in one or more free spaces 2530 to solidify said drug-containing plasticized matrix 2522 and form drug-containing solid 2523 attached to said three dimensional structural framework of one or more fibers 2505 (e.g., drug-containing solid 2523 attached to said three dimensional structural framework of one or more fibers 2505 within one or more free spaces 2530 of said supporting solid 2500).

In some embodiments, an amount (e.g., the mass, weight, etc.) of drug-containing plasticized matrix added to a support structure may be precisely controlled. By way of example but not by way of limitation, if the composition (e.g., the density, etc.) of a drug-containing plasticized matrix is uniform, the amount (e.g., the mass, weight, etc.) of drug-containing plasticized matrix added to a support structure may be controlled by controlling the volume of drug-containing plasticized matrix added to said support structure.

In some embodiments, therefore, a supporting solid may be placed (e.g., positioned, disposed, located, etc.) in a closed or substantially closed mold (e.g., a cavity surrounded by walls, a housing substantially surrounding said supporting solid, a wall substantially surrounding said supporting solid, etc.). Said closed or substantially closed mold may have an internal volume greater than the volume occupied by a solid structure (e.g., a three dimensional support structure, etc.) of said supporting solid. If the internal volume of the mold and the volume of said solid structure (e.g., the volume occupied by said solid structure within said mold, etc.) are controlled, the free volume within said mold may be controlled, too.

In preferred embodiments, said mold may have at least an opening towards an extrusion channel. Thus, upon extruding drug-containing plasticized matrix through said opening, said drug-containing plasticized matrix may fill free volume between the internal volume of said mold and the volume occupied by said solid structure within said mold.

In preferred embodiments, an entire or a substantial part of (e.g., a substantial fraction of) the free volume between the internal volume of said mold and the volume occupied by said solid structure may be filled with drug-containing plasticized matrix. Thus, if the free volume within said mold is controlled, the volume of drug-containing plasticized matrix that is filled into the mold may be controlled, too.

In some embodiments, free volume may comprise free space within an exterior or outer volume of a supporting solid.

In some embodiments, moreover, an exterior or outer volume of a supporting solid may be about or substantially the same as an internal volume of a mold, and only (or predominantly) free space within the exterior volume of said supporting solid may be filled with drug-containing plasticized matrix.

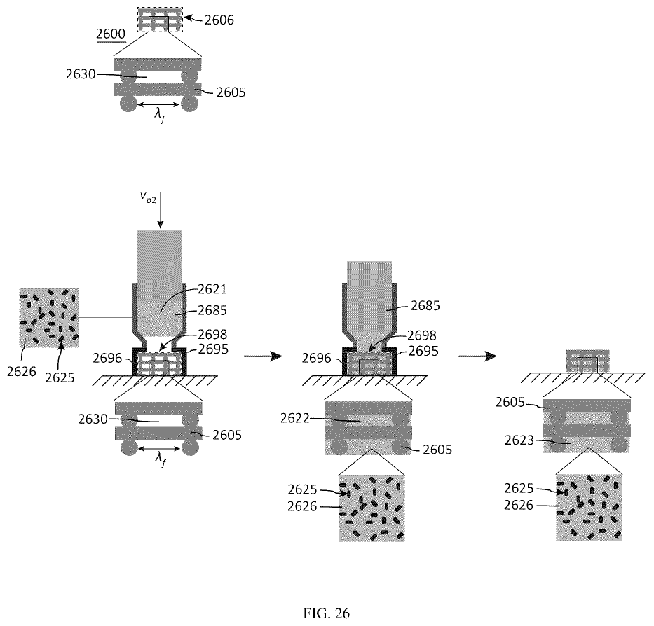

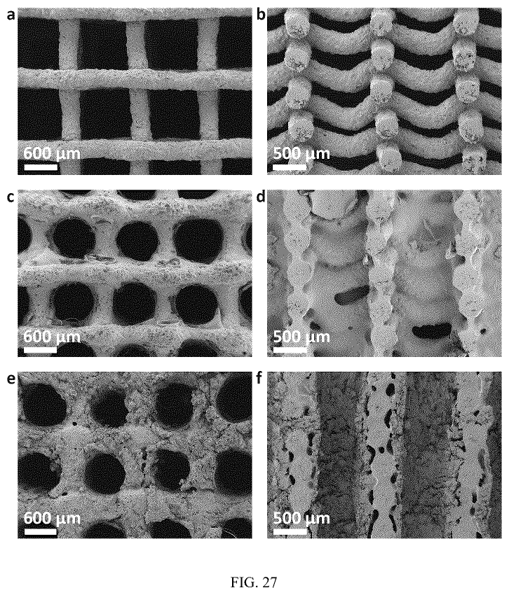

FIG. 26 presents another non-limiting method or way of adding a controlled amount of drug-containing plasticized matrix 2621,2622 to a support structure 2605. A supporting solid 2600 comprising a controlled volume of free space 2630 within its exterior volume may be disposed in a substantially closed mold 2695. Said mold 2695 may have an internal volume 2696 substantially the same as the exterior volume of the supporting solid 2600. Said mold 2695 may further have an opening 2698 towards an extrusion channel 2685 filled with drug-containing plasticized matrix 2621. Said drug-containing plasticized matrix 2621,2622 may then be extruded through said extrusion channel 2685 by application of mechanical work. Also, a controlled amount of said extruded drug-containing plasticized matrix 2622 may be extruded (e.g., transported, squeezed, filled, etc.) into one or more internal, open free spaces 2630 within said exterior volume of said supporting solid 2600 and added to said support structure 2605 by application of mechanical work. Eventually, the content in the internal volume 2605,2622,2696 of the mold 2695 may be removed (e.g., ejected, etc.) and solidified to form a drug-containing solid 2623 attached to said supporting solid 2605.

In some embodiments, mechanical work may be applied on a drug-containing plasticized matrix by one or more rotating screws. Similarly, in some embodiments, mechanical work may be applied on a drug-containing plasticized matrix by an advancing piston. It may be obvious to a person of ordinary skill in the art that more ways may exist to apply mechanical work on a drug-containing plasticized matrix for extrusion through an extrusion channel and/or for extrusion through an opening of a mold and/or for extrusion into one or more free spaces within an exterior volume of a supporting solid and/or for adding to supporting solid.

In some embodiments, moreover, a piston that fills drug-containing plasticized matrix into a closed or substantially closed mold may further be equipped with a force or pressure sensor. The force or pressure required to advance the piston may increase if the mold is full. If the force or pressure is too large, the piston may cease to advance.

In some embodiments, moreover, for enhancing the bond strength between a supporting solid and an added drug-containing solid, at least one second solvent solvating a second excipient in a drug-containing plasticized matrix may also solvate a first excipient in a supporting solid. Thus, in some embodiments, a first excipient may be soluble in and/or be plasticized by and/or by solvated by a second solvent solvating at least a second excipient.

In some embodiments, at least one solvent includes dimethyl sulfoxide (DMSO). Similarly, in some embodiments, at least one solvent includes dimethylformamide.

In some embodiments, second solvent may be evaporated or boiled away from drug-containing plasticized matrix added to a support structure by reducing the pressure (e.g., by reducing the pressure of the gas) surrounding the added drug-containing plasticized matrix to a pressure about equal to or slightly greater or lower (e.g., smaller) than the vapor pressure of the second solvent (e.g., the vapor pressure of the second solvent at the temperature of the second solvent in the added drug-containing plasticized matrix).

In some embodiments, the pressure of the gas surrounding the added drug-containing plasticized matrix during second solvent removal may be less than the atmospheric pressure (e.g., less than 1 bar). This includes, but is not limited to a pressure of the gas surrounding the added drug-containing plasticized matrix during second solvent removal no greater than 800 mbar, or no greater than 700 mbar, or no greater than 600 mbar, or no greater than 500 mbar, or no greater than 400 mbar, or no greater than 300 mbar, or no greater than 200 mbar, or no greater than 100 mbar, or no greater than 75 mbar.

In some embodiments, moreover, a reduced pressure (or vacuum) may be maintained for prolonged time, such as longer than 1 minute, or longer than 2 minutes, or longer than 5 minutes, or longer than 10 minutes, or longer than 20 minutes, or longer than 50 minutes, or longer than 100 minutes, or longer than 200 minutes, or longer than 500 minutes, or longer than 1000 minutes. Such “vacuum drying” or removing second solvent using a vacuum may allow more uniform shrinkage of the added drug-containing plasticized matrix. Moreover, it may allow drying at lower temperatures.

It may be obvious to a person of ordinary skill in the art that more ways of evaporating second solvent from a drug-containing plasticized matrix exist. Such ways include, but are not limited to blowing a gas, such as air, on or through the drug-containing plasticized matrix, and so on. All these methods of second solvent evaporation that are obvious to a person of ordinary skill in the art are included in this invention.

Moreover, it may be obvious to a person of ordinary skill in the art that more ways of adding drug-containing matter to support structures exist. All such methods obvious to a person of ordinary skill in the art are included in this invention.

(d) Embodiments Related to Pushing a Needle or Pin into Drug-Containing Matter to Form One or More Channels

In some embodiments herein, a method of manufacturing pharmaceutical dosage forms as disclosed herein may comprise or further comprise pushing a needle or pin into or through drug-containing matter to form one or more channels within said drug-containing matter.

In some embodiments, one or more channels may have at least one open end contiguous with and terminating at the exterior or outer surface of a drug-containing matter (e.g., a drug-containing plasticized matrix, a solidifying drug-containing plasticized matrix, a solidified drug-containing plasticized matrix, a drug-containing solid, etc). In some embodiments, moreover, one or more channels may have at least two open ends contiguous with and terminating at the exterior or outer surface of a drug-containing matter.

In some embodiments, furthermore, one or more channels may be substantially straight. Also, in some embodiments, one or more channels may comprise a substantially uniform cross section along their length. In some embodiments, furthermore, one or more channels may comprise a substantially uniform cross section within a drug-containing matter.

In some embodiments, moreover, a plurality of channels may be substantially parallel to each other. By way of example but not by way of limitation, in some embodiments, at least two channels may be substantially parallel to each other. This includes, but is not limited to a plurality of at least three channels, or at least four channels, or at least five channels, or at least six channels, etc. being substantially parallel to each other.

In some embodiments, furthermore, a plurality of channels may be substantially orderly arranged within a drug-containing matter (e.g., a drug-containing plasticized matrix, a solidifying drug-containing plasticized matrix, a solidified drug-containing plasticized matrix, a drug-containing solid, drug-containing matter added to an internal, three dimensional support structure, one or more drug-containing plasticized matrices or drug-containing solids added or attached to an internal, three dimensional support structure, etc.). Similarly, in some embodiments, a plurality of channels may be arranged in a substantially ordered pattern within drug-containing matter (e.g., drug-containing plasticized matrix, solidifying drug-containing plasticized matrix, solidified drug-containing plasticized matrix, drug-containing solid, drug-containing matter added or attached to an internal, three dimensional support structure, one or more drug-containing plasticized matrices or drug-containing solids added or attached to an internal, three dimensional support structure, etc.). By way of example but not by way of limitation, in some embodiments a plurality of channels may be arranged in a substantially square lattice (as shown, for example, in the non-limiting top view of FIG. 7).

In some embodiments, a drug-containing solid (e.g., a drug-containing solid added or attached to a support structure) produced herein comprises an average thickness no greater than 6 mm (e.g., no greater than 5.5 mm, no greater than 5 mm, no greater than 4.5 mm, no greater than 4 mm, no greater than 3.5 mm, etc.).

The thickness of a drug-containing solid may, however, also not be too small. In some embodiments, therefore, a drug-containing solid (e.g., a drug-containing solid added or attached to a support structure) produced herein comprises an average thickness greater than 5 Wm. Preferably, however, a drug-containing solid (e.g., a drug-containing solid added or attached to a support structure) produced herein may comprise an average thickness greater than 10 Wm, and even more preferably greater than 25 Wm, and even more preferably greater than 50 μm, and even more preferably greater than 75 μm.

In some embodiments, moreover, a thickness or average thickness of a drug-containing solid is in the ranges 10 μm-5 mm, 20 μm-5 mm, 25 μm-4 mm, 30 μm-3 mm, 20 μm-3.5 mm, 25 μm-5 mm, 25 μm-2.5 mm, 25 μm-1.5 mm, 30 μm-2 mm, 30 μm-1.5 mm, 40 μm-3 mm, 10 μm-1.5 mm, or 50 μm-2.5 mm.

It may be obvious to a person of ordinary skill in the art that more ways of manufacturing pharmaceutical solid dosage forms herein exist. All such methods of manufacturing pharmaceutical solid dosage forms obvious to a person of ordinary skill in the art are included in this invention.

### EXPERIMENTAL EXAMPLES

The following examples present ways by which the disclosed dosage forms may be prepared and analyzed, and may enable one of skill in the art to more readily understand the principle thereof. The examples also include ways for preparing and analyzing particulate dosage forms. The examples are presented by way of illustration and are not meant to be limiting in any way.

Because the non-limiting experimental dosage forms prepared herein comprise three-dimensional structural frameworks of fibers as support structures, they are also referred to herein as “fibrous dosage forms”.

### Example 1: Preparation of Fibrous Dosage Forms

### (a) Materials for Preparing Fibrous Dosage Forms

The materials used for preparing the fibrous dosage forms were as follows.

Excipients in the fiber core: Hydroxypropyl methylcellulose with a molecular weight of 120 kg/mol (HPMC2), purchased from Merck KGaA, Darmstadt, Germany; methacrylic acid-ethyl acrylate copolymer (1:1), with a molecular weight of about 250 kg/mol (trade name: Eudragit L100-55), received from Evonik, Essen, Germany.

Contrast agent in the fiber core: Barium sulfate (BaSO₄), purchased as solid particles of size ˜1 μm, from Humco, Austin, TX.

Fiber-strengthening coating: Methacrylic acid-ethyl acrylate copolymer as in the fiber core.

Excipients in the inter-fiber formulation: Hydroxypropyl methylcellulose with a molecular weight of 10 kg/mol (HPMC1), purchased from Merck KGaA, Darmstadt, Germany; and methacrylic acid-ethyl acrylate copolymer as in the fiber core.

Drug: Nilotinib hydrochloride monohydrate, purchased as solid particles from the European Directorate for the Quality of Medicine (EDQM), Strasbourg, France.

Solvents: Dimethylsulfoxide (DMSO) and acetone.

### (b) Preparation of Uncoated Fibrous Cylindrical Disks (Fiber Core)

First, solid particles of HPMC2, Eudragit L100-55, and barium sulfate were mixed with liquid DMSO to form a uniform suspension. The concentrations of HPMC2, Eudragit L100-55, and barium sulfate were 500, 300, and 343 mg/ml of DMSO.

The suspension was extruded through a laboratory extruder to form a uniform viscous paste. The viscous paste was put in a syringe equipped with a hypodermic needle of inner radius, Rₙ=205 μm. The paste was extruded through the needle to form a wet fiber that was patterned layer-by-layer in a cross-ply structure. The nominal inter-fiber spacing was 1500 μm and the number of layers was 32.

After patterning, the solvent was evaporated by blowing warm air at about 50° C. and a velocity of about 1 m/s over the structure for a day. Finally, a cylindrical disk with nominal diameter 14 mm and thickness 8 mm was punched out.

The solid fibrous cylindrical disk consisted 43.75 wt % HPMC2, 26.25 wt % Eudragit L100-55, and 30 wt % barium sulfate, Table 3 later.

The solid fibrous cylindrical disk consisted 43.75 wt % HPMC2, 26.25 wt % Eudragit L100-55, and 30 wt % barium sulfate, Table 1 later.

It may be noted that the solid fibrous cylindrical disks may generally be understood herein as “support structure”.

### (c) Coating the Fibers of the Fibrous Cylindrical Disks

The cylindrical disks so produced were dip-coated with a fiber-strengthening, enteric coating solution. The coating solution consisted of Eudragit L100-55 and acetone at a polymer concentration of 100 mg/ml. The coating was applied by dipping the disks into the coating solution for about 5-10 seconds. The coated disks were then put in a vacuum chamber held at about 35° C. To evaporate the solvent the pressure was slowly reduced from atmospheric to 200 Pa, and maintained at this value for about two hours. After solvent evaporation, the inter-fiber spaces were opened by pushing a 0.8 mm diameter needle through them. The dipping-evaporation process was executed three times.

It may be noted that the solid fibrous cylindrical disks may generally be understood herein as “coated support structure”.

(d) Filling the Spaces Between Coated Fibers with Drug

First, solid particles of nilotinib, HPMC1, and Eudragit L100-55 were mixed with liquid DMSO to form a uniform dispersion. The weight fractions of nilotinib, HPMC1, Eudragit L100-55, and DMSO in the dispersion were 0.372, 0.223, 0.025, and 0.38, respectively.

The coated cylindrical disk was then introduced in a cylindrical mold with a diameter of 14 mm. Subsequently, about 540 mg of the dispersion was dispensed on the disk and pressed into the inter-fiber spaces with a piston. After filling, circular channels were formed in the inter-fiber spaces by pushing a 0.72 mm diameter needle through them. To evaporate the solvent and solidify the dispersion the sample was put in a vacuum chamber maintained at a pressure of 200 Pa and a temperature of 20° C. for about a day.

After solvent evaporation, the composition of the inter-fiber space was 60 wt % nilotinib, 36 wt % HPMC1, and 4 wt % Eudragit L100-55, Table 1.

It may be noted, moreover, that after solvent evaporation drug-containing solid in the inter-fiber spaces is attached to the coated cylindrical disk.

After solvent evaporation, preparation of the non-limiting experimental fibrous dosage forms herein was final. It may be obvious to a person of ordinary skill in the art, however, that additional steps could be added to prepare dosage forms with desirable properties. Any such additional steps obvious to a person of ordinary skill in the art are all included in this invention.

### Example 2: Microstructures and Weights of Fibrous Disks and Fibrous Dosage Forms

The microstructures of uncoated fibrous cylindrical disks (uncoated fiber cores), coated fibrous cylindrical disks (coated fiber cores), and “final” dosage forms were imaged by a Zeiss Merlin High Resolution SEM with a GEMINI column. The top surfaces of the fibrous structures and gastroretentive dosage forms were imaged after coating the sample with a 10-nm thick layer of gold. The longitudinal sections were imaged after the sample was cut with a thin blade (MX35 Ultra, Thermo Scientific, Waltham, MA) and coated with gold as above. All specimens were imaged with an in-lens secondary electron detector. The accelerating voltage was 5 kV, and the probe current 95 pA.

The weights of the uncoated fibrous cylindrical disks, the coated fibrous cylindrical disks, and the final dosage forms were measured by an analytical balance with a resolution of 0.1 mg (Mettler Toledo, Greifensee, Switzerland).

FIGS. 27a and 27b show the top and longitudinal sectional views of the microstructure of an uncoated fibrous cylindrical disk. The fiber radius, R_{f,0}=165 μm, and the inter-fiber spacing, λ₀=1298 μm, Table 1. The weight, or mass, of the uncoated fibrous cylindrical disk was 500 mg, Table 1.

The microstructure of a coated fibrous cylindrical disk is shown in FIGS. 27c and 27d. The coating surrounded the fibers, and bridged the neighboring fibers vertically, but not horizontally. Thus, the microstructure of the coated fibrous cylindrical disk may be approximated as having vertical walls of thickness, 2R_{f,0}, and vertical square channels of width, λ₀−2R_{f,0}. The weight of the coated fibrous cylindrical disk was 650 mg, Table 1.

FIGS. 27e and 27f are the images of the “final” dosage forms with coated fibers and drug-filled inter-fiber space. At the center of the inter-fiber space the dosage forms had an open, cylindrical channel with radius, R_{c,0}=302 μm. Between the channel and the coated fibers was a drug-loaded annulus with wall thickness, h_{a,0}≈156 μm. The weight of the final dosage form was 984 mg, Table 1.

Several additional parameters may be obtained as follows. The volume fraction of fibers in the dosage form may be written as:

$${φ}_{f} = \frac{{V}_{f}}{{V}_{df}} = \frac{{M}_{f}}{{ρ}_{f}} \times \frac{1}{π {R}_{df , 0}^{2} {H}_{0}} \qquad ( 1 )$$

where V_{f} is the volume of fibers in the dosage form, V_{df} the volume of the dosage form, M_{f} the mass of the uncoated fibrous cylindrical disk, ρ_{f} the density of the solid fibers, R_{df,0} the radius of the solid fibrous dosage form, and H₀ its thickness. Substituting the relevant parameters listed in Table 1 and in Eq. (1), φ_{f}=0.3.

Similarly, the volume fraction of enteric coating in the dosage form is:

$${φ}_{e c} = \frac{{V}_{ec}}{{V}_{df}} = \frac{{M}_{e c} - {M}_{f}}{{ρ}_{e c}} \times \frac{1}{π {R}_{df , 0}^{2} {H}_{0}} \qquad ( 2 )$$

where V_{ec} is the volume of enteric coating in the dosage form, M_{ec} the mass of the coated fibrous cylindrical disk, and ρ_{ec} the density of the solid coating. For the relevant parameters listed in Table 1, by Eq. (2) φ_{ec}=0.15.

The drug mass in the dosage form may be obtained as:

$${M}_{d , 0} = {w}_{d , a} ( {M}_{df} - {M}_{ec} ) \qquad ( 3 )$$

TABLE 1
Selected microstructural parameters and composition of the
non-limiting experimental fibrous dosage forms

| Symbol | Description | Value | |
|---|---|---|---|
| H₀ | thickness of solid dosage form | 8 | mm |
| h_{α,0} | thickness of drug-loaded annulus | 156 | μm ^{a} |
| M_{d,0} | mass of drug in dosage form | 200 | mg ^{b} |
| M_{dƒ} | mass of final dosage form | 984 | mg ^{c} |
| M_{ec} | mass of enteric coated fibrous cylindrical disk | 650 | mg ^{c} |
| M_{ƒ} | mass of uncoated fibrous cylindrical disk | 500 | mg ^{c} |
| n_{α} or n_{c} | number of annuli or channels in dosage form | 91.4 ^{d} | |
| nₗ | number of fiber layers in dosage form | 32 | |
| R_{c,0} | radius of inner circle of drug-containing annulus | 302 ± 32 | μm ^{a} |
| R_{df,0} | radius of dosage form | 7 | mm |
| R_{f,0} | fiber radius | 165 ± 6 | μm ^{a} |
| W_{d,α} | weight fraction of drug in annulus | | |
| W_{HPMC1,α} | weight fraction of low-molecular-weight HPMC (HPMC1) in annulus | 0.36 | |
| W_{ee,α} | weight fraction of enteric excipient (Eudragit L100-55) in annulus | 0.04 | |
| W_{HPMC2,ƒ} | weight fraction of high-molecular-weight HPMC (HPMC2) in fiber core | 0.6 | |
| W_{ee,ƒ} | weight fraction of enteric excipient in fiber core | 0.36 | |
| W_{BαS04ƒ} | weight fraction of BaSO₄ in fiber core | 0.04 | |
| λ₀ | inter-fiber spacing | 1298 ± 54 | μm |

| | | |
|---|---|---|
| φ_{ec} | volume fraction of enteric coating in the dosage form | 0.15 ^{e} |
| φ_{ƒ} | volume fraction of solid fiber core in dosage form | 0.3 ^{f} |

^{a} From the scanning electron micrographs shown in FIG. 16.
^{b} From Eq. (3)
^{c} From weight measurements.
^{d} Calculated as n_{α} = n_{c} = πR_{dƒ,0}²/λ₀².
^{e} From Eq. (2) using a density of the enteric coating, ρ_{ec} = 800 kg/m³.
^{f} From Eq. (1) using a density of the solid fibers, ρ_{ƒ} = 1367 kg/m³.

where w_{d,a} is the weight fraction of drug in the annuli and M_{df} the mass of the final dosage form. For the relevant parameters (w_{d,a}=0.6, M_{df}=984 mg, and M_{ec}=650 mg), by Eq. (3) M_{d,0}=200 mg, Table 1.

The following claims are suggestive and may be considered non-limiting.

We claim:

1. A method of manufacturing pharmaceutical solid dosage forms comprising the steps of:
preparing a supporting three-dimensional structural framework of one or more fibers, said one or more fibers comprising at least a first pharmaceutical excipient;
forming a drug-containing plasticized matrix, said drug-containing plasticized matrix disposed in an extrusion channel and comprising at least one drug and at least one second pharmaceutical excipient;
extruding said drug-containing plasticized matrix through said extrusion channel by application of mechanical work;
filling a controlled amount of said extruded drug-containing plasticized matrix into one or more free spaces between fibers or fiber segments of said supporting fiber structural framework; and
solidifying said drug-containing plasticized matrix within said one or more free spaces to form drug-containing solid attached to said three-dimensional fiber structural framework.

2. The method of claim 1, wherein preparing the supporting three-dimensional structural framework of one or more fibers comprises the steps of:
forming a plasticized matrix comprising at least a first pharmaceutical excipient, said plasticized matrix disposed in an extrusion channel;
extruding said plasticized matrix through a fiber fabrication exit port of said extrusion channel by application of mechanical work to form extruded plasticized fiber;
depositing said extruded plasticized fiber onto a fiber assembling stage to form a three-dimensional fiber structural framework defined by the motion of said stage; and
solidifying said three-dimensional fiber structural framework.

3. The method of claim 2, wherein the plasticized matrix comprising at least a first pharmaceutical excipient is formed by heating a composition comprising at least one first pharmaceutical excipient.

4. The method of claim 2, wherein the plasticized matrix comprising at least a first pharmaceutical excipient is formed by mixing at least a first pharmaceutical excipient and at least a first solvent solvating said first excipient.

5. The method of claim 2, wherein mechanical work to extrude plasticized matrix through an exit port or a fiber fabrication exit port is applied on said plasticized matrix by one or more rotating screws.

6. The method of claim 2, wherein mechanical work to extrude plasticized matrix through an exit port or fiber fabrication exit port is applied on said plasticized matrix by an advancing piston.

7. The method of claim 2, wherein plasticized matrix is extruded through an exit port or a fiber fabrication exit port at a controlled speed.

8. The method of claim 2, wherein plasticized matrix is extruded through an exit port or a fiber fabrication exit port at a speed determined by the speed of advancement of a piston.

9. The method of claim 2, wherein extruded plasticized fiber or deposited three-dimensional fiber structural framework is solidified by cooling or by evaporating at least a first solvent.

10. The method of claim 1, wherein the drug-containing plasticized matrix is formed by heating a composition comprising at least one second pharmaceutical excipient.

11. The method of claim 1, wherein the drug-containing plasticized matrix is formed by mixing at least a second pharmaceutical excipient and at least a second solvent solvating said second excipient.

12. The method of claim 1, wherein mechanical work to extrude drug-containing plasticized matrix through said extrusion channel is applied on said plasticized matrix by one or more rotating screws.

13. The method of claim 1, wherein mechanical work to extrude drug-containing plasticized matrix through said extrusion channel is applied on said plasticized matrix by an advancing piston.

14. The method of claim 1, wherein drug-containing plasticized matrix is extruded through said extrusion channel at a controlled speed.

15. The method of claim 1, wherein drug-containing plasticized matrix is extruded through said extrusion channel at a speed determined by the speed of advancement of a piston.

16. The method of claim 1, wherein drug-containing plasticized matrix is solidified by cooling or by evaporating at least a second solvent.

17. The method of claim 1, further comprising the step of coating said supporting three-dimensional structural framework of one or more fibers or a part thereof with a coating, said coating comprising at least a third pharmaceutical excipient.

18. The method of claim 1, further comprising the step of pushing a needle or pin into or through drug-containing matter to form one or more channels within said drug-containing matter.

19. The method of any preceding claim, wherein the three-dimensional structural framework of one or more fibers is prepared by 3D-micro-patterning or 3D-printing.

20. A method of manufacturing pharmaceutical solid dosage forms comprising the steps of:
preparing a supporting three-dimensional structural framework of one or more fibers, said one or more fibers comprising at least a first pharmaceutical excipient; and
adding or attaching a controlled amount of drug-containing matter to said supporting three-dimensional structural framework;
wherein the step of preparing the supporting comprises the steps of:
forming a plasticized matrix comprising at least a first pharmaceutical excipient, said plasticized matrix disposed in an extrusion channel;
extruding said plasticized matrix through a fiber fabrication exit port of said extrusion channel by application of mechanical work to form extruded plasticized fiber;
depositing said extruded plasticized fiber onto a fiber assembling stage to form a three-dimensional fiber structural framework defined by the motion of said stage; and
solidifying said three-dimensional fiber structural framework;
and wherein the step of adding or attaching a controlled amount of drug-containing matter to said supporting three-dimensional structural framework comprises the steps of:
forming a drug-containing plasticized matrix, said drug-containing plasticized matrix disposed in an extrusion channel and comprising at least one drug and at least one second pharmaceutical excipient;
extruding said drug-containing plasticized matrix through said extrusion channel by application of mechanical work;
filling a controlled amount of said extruded drug-containing plasticized matrix into one or more free spaces between fibers or fiber segments of said supporting fiber structural framework; and
solidifying said drug-containing plasticized matrix within said one or more free spaces to form drug-containing solid attached to said three-dimensional fiber structural framework.
